(19) United States
(12) Patent Application Publication
(10) Pub. No.: US 2013/0029906 A1
(43) Pub. Date: Jan. 31, 2013

(54) METHOD OF REGULATING PPAR, OBESITY RELATED PATHWAYS AND THEIR ASSOCIATED METABOLIC IMPACT

(71) Applicants: Eric Simard, Rosemère (CA); Eric Trottier, Mirabel (CA); Josée Beaulieu, Laval (CA); Jean-Francois Lapointe, Laval (CA); Pierre Lemieux, Sainte Therese (CA)

(72) Inventors: Eric Simard, Rosemère (CA); Eric Trottier, Mirabel (CA); Josée Beaulieu, Laval (CA); Jean-Francois Lapointe, Laval (CA); Pierre Lemieux, Sainte Therese (CA)

(21) Appl. No.: 13/513,442

(22) PCT Filed: Dec. 3, 2010
(86) PCT No.: PCT/CA10/01928

§ 371 Date: Sep. 17, 2012

Related U.S. Application Data
(60) Provisional application No. 61/266,580, filed on Dec. 4, 2009.

(51) Int. Cl. A61K 38/01; A61P 3/08

(52) U.S. Cl. 514/6.8; 514/1.1

(57) ABSTRACT

It is disclosed a method of modulating a peroxisome proliferator activated receptor (PPAR) activity in a patient comprising administering to said subject an effective amount of a malleable protein matrix (MPM), uses thereof and pharmaceutical compositions comprising MPM for treating obesity related disorders and their associated metabolic impact by modulating PPAR activity.

### TECHNICAL FIELD

The present description relates to a method of regulating nuclear receptor mediated processes in a subject comprising administering to said subject an effective amount of a malleable protein matrix (MPM) modulating the peroxisome proliferator activated receptors (PPARs) and some biological pathway associated to obesity.

### BACKGROUND

Obesity has reached epidemic proportions globally with more than 1 billion adults overweight, at least 300 million of them clinical obese. Recent statistics by the Center for Disease Control (“CDC”) estimate that approximately 65% of all Americans are overweight or obese. It is also increasing steadily in developing countries and is affecting an ever younger population. Obesity is responsible for more than 300,000 deaths annually, and will soon overtake tobacco usage as the primary cause of preventable death in the United States. Obesity is a chronic disease that contributes directly to numerous dangerous co-morbidities, including type 2 diabetes, cardiometabolic diseases, hepatic disorders, cardiovascular disease, inflammatory diseases, premature aging, and some forms of cancer. Low levels of physical activity, sedentary lifestyles, stress, depression and consumption of high-fat and fast foods are responsible for unwanted weight gain.

Obesity is a chronic disorder of energy imbalance characterized by an excess of energy intake in the long term compared with limited energy expenditure, leading to storage of the excess energy in the form of adipose tissue.

Adipose tissue consists primarily of adipocytes. Vertebrates possess two distinct types of adipose tissue: white adipose tissue (WAT) and brown adipose tissue (BAT). WAT stores and releases fat according to the nutritional needs of the animal. This stored fat is used by the body for (1) heat insulation (e.g., subcutaneous fat), (2) mechanical cushion (e.g., surrounding internal organs), and (3) as a source of energy. BAT burns fat, releasing the energy as heat through thermogenesis. BAT thermogenesis is used both (1) to maintain homeothermy by increasing thermogenesis in response to lower temperatures and (2) to maintain energy balance by increasing energy expenditure in response to increases in caloric intake (Sears et al., 1996, Mol. Cell. Biol. 16(7): 3410-3419). BAT is also the major site of thermogenesis in rodents and plays an important role in thermogenesis in human infants. In humans, and to lesser extent rodents, brown fat diminishes with age, but can be re-activated under certain conditions, such as prolonged exposure to cold, maintenance on a high fat diet and in the presence of noradrenaline producing tumors.

Fat metabolism is regulated by two pathways, lipogenesis and lipolysis. Lipogenesis is the deposition of fat which occurs in the liver and in adipose tissue at cytoplasmic and mitochondrial sites. This process allows the storage of energy that is ingested which is not needed for current energy demands. Lipolysis is the chemical decomposition and release of fat from adipose and/or other tissues. This process predominates over lipogenesis when additional energy is required by the body.

Obesity is a well-established risk factor for the development of insulin resistance, of dyslipidaemia and, ultimately, of non-insulin-dependent diabetes.

Diabetes mellitus, commonly called diabetes, refers to a disease process derived from multiple causative factors and characterized by elevated levels of plasma glucose, referred to as hyperglycemia. According to the American Diabetes Association, diabetes mellitus is estimated to affect approximately 6% of the world population. Uncontrolled hyperglycemia is associated with increased and premature mortality due to an increased risk for microvascular and macrovascular diseases, including nephropathy, neuropathy, retinopathy, hypertension, cerebrovascular disease, coronary heart disease, and other cardiovascular diseases. Therefore, control of glucose homeostasis is a critically important approach for the treatment of diabetes.

There are two major forms of diabetes: type 1 diabetes (formerly referred to as insulin-dependent diabetes or IDDM); and type 2 diabetes (formerly referred to as non-insulin dependent diabetes or NIDDM).

Type 1 diabetes is the result of an absolute deficiency of insulin, the hormone which regulates glucose utilization. This insulin deficiency is usually characterized by β cell destruction within the Islets of Langerhans in the pancreas, which usually leads to absolute insulin deficiency. Type 1 diabetes has two forms: Immune-Mediated Diabetes Mellitus, which results from a cellular mediated autoimmune destruction of the β cells of the pancreas; and Idiopathic Diabetes Mellitus, which refers to forms of the disease that have no known etiologies.

Type 2 diabetes is a complex disease characterized by defects in glucose and lipid metabolism. Typically there are perturbations in many metabolic parameters including increases in fasting plasma glucose levels, free fatty acid levels and triglyceride levels (hypertriglyceridemia), as well as a decrease in the ratio of HDL/LDL. One of the principal underlying causes of diabetes is thought to be when muscle, fat and liver cells fail to respond to normal concentrations of insulin (insulin resistance). Insulin resistance may be due to reduced numbers of insulin receptors on these cells, or a dysfunction of signaling pathways within the cells, or both. Insulin resistance is characteristically accompanied by a relative, rather than absolute, insulin deficiency. Type 2 diabetes can range from predominant insulin resistance with relative insulin deficiency to predominant insulin deficiency with some insulin resistance.

The β cells in insulin resistant individuals initially compensate for this insulin resistance by secreting abnormally high amounts of insulin (hyperinsulemia). Over time, these cells become unable to produce enough insulin to maintain normal glucose levels, indicating progression to type 2 diabetes. When inadequate amounts of insulin are present to compensate for insulin resistance and adequately control glucose, a state of impaired glucose tolerance develops. In a significant number of individuals, insulin secretion declines further and the plasma glucose level rises, resulting in the clinical state of diabetes. Type 2 diabetes can be due to a profound resistance to insulin stimulating regulatory effects on glucose and lipid metabolism in the main insulin-sensitive tissues: muscle, liver and adipose tissue. This resistance to insulin responsiveness results in insufficient insulin activation of glucose uptake, oxidation and storage in muscle and inadequate insulin repression of lipolysis in adipose tissue and of glucose production and secretion in liver. In type 2 diabetes, free fatty acid levels are often elevated in obese and some non-obese patients and lipid oxidation is increased.

Type 2 diabetes is brought on by a combination of genetic and acquired risk factors, including a high-fat diet, lack of exercise, and aging. Worldwide, type 2 diabetes has become an epidemic, driven by increases in obesity and a sedentary lifestyle, widespread adoption of western dietary habits, and the general aging of the population in many countries. In 1985, an estimated 30 million people worldwide had diabetes. By 2000, this figure had increased 5-fold, to an estimated 154 million people. The number of people with diabetes is expected to double between now and 2025, to about 300 million.

Therapies aimed at reducing peripheral insulin resistance are available. The most relevant to this invention are drugs of the thiazolidinedione (TZD) class namely troglitazone, pioglitazone, and rosiglitazone. In the US these have been marketed under the names Rezulin™, Avandia™ and Actos™, respectively. The principal effect of these drugs is to improve glucose homeostasis. Notably in diabetics treated with TZDs there are increases in peripheral glucose disposal rates indicative of increased insulin sensitivity in both muscle and fat.

Premature development of atherosclerosis and increased rate of cardiovascular and peripheral vascular diseases are characteristic features of patients with diabetes, with hyperlipidemia being an important precipitating factor for these diseases.

Coronary heart disease (CHD) is the major cause of death in Type 2 diabetic and metabolic syndrome patients (i.e., patients that fall within the “deadly quartet” category of impaired glucose tolerance, insulin resistance, hypertriglyceridaemia and/or obesity). Many important factors are known to increase CHD risk in obesity, like the elevated plasma levels of the plasminogen activator inhibitor-1 (PAI-1) or the LDL-cholesterol. Accordingly, agents that inhibit PAI-1 would be of utility in treating conditions originating from fibrinolytic disorder such as deep vein thrombosis, coronary heart disease, pulmonary fibrosis, polycystic ovary syndrome, etc. However, agent able to reduce several factors at once would be much more interesting for the treatment of obesity.

The metabolic syndrome is a major global health problem, involving several factors, associated with weight gain. In the US, the prevalence in the adult population is currently estimated to be approximately 25%, and it continues to increase both in the US and worldwide. The metabolic syndrome is characterized by a combination of insulin resistance, hypertension, obesity and dyslipidemia leading to increased morbidity and mortality of cardiovascular diseases. People with the metabolic syndrome are at increased risk of developing type 2 diabetes, hyperlipidemia, hypercholesterolemia or dyslipidemia.

Hyperlipidemia is a condition generally characterized by an abnormal increase in serum lipids in the bloodstream and, as noted above, is an important risk factor in developing atherosclerosis and coronary heart disease. For a review of disorders of lipid metabolism, see, e.g., Wilson, J. et al., (ed.), Disorders of Lipid Metabolism, Chapter 23, Textbook of Endocrinology, 9th Edition, (W.B. Sanders Company, Philadelphia, Pa. U.S.A. 1998; this reference and all references cited therein are herein incorporated by reference). Serum lipoproteins are the carriers for lipids in the circulation. They are classified according to their density: chylomicrons; very low-density lipoproteins (VLDL); intermediate density lipoproteins (IDL); low density lipoproteins (LDL); and high density lipoproteins (HDL). Hyperlipidemia is usually classified as primary or secondary hyperlipidemia. Primary hyperlipidemia is generally caused by genetic defects, while secondary hyperlipidemia is generally caused by other factors, such as various disease states, drugs, and dietary factors. Alternatively, hyperlipidemia can result from both a combination of primary and secondary causes of hyperlipidemia.

Hypercholesterolemia, a form of hyperlipidemia, is characterized by excessive high levels of blood cholesterol. The blood cholesterol pool is generally dependant on dietary uptake of cholesterol from the intestine and biosynthesis of cholesterol throughout the body, especially the liver. The majority of the cholesterol in plasma is carried on apolipoprotein B-containing lipoproteins, such as the very-low-density lipoproteins (VLDL), low-density lipoproteins (LDL), intermediate density lipoproteins (IDL) and high density lipoproteins (HDL). Hypercholesterolemia is characterized by elevated LDL cholesterol levels. The risk of coronary artery disease in man increases when LDL and VLDL levels increase. Conversely, high HDL levels are protective against coronary artery disease (see Gordon and Rifkind, N. Engl. J. Med., 1989, 321: 1311-15; and Stein and Stein, Atherosclerosis, 1999 144: 285-303). Therefore, although it is desirable to lower elevated levels of LDL, it is also desirable to increase HDL levels.

Initial treatment for hypercholesterolemia is to place the patients on a low fat/low cholesterol diet coupled with adequate physical exercise, followed by drug therapy when LDL-lowering goals are not met by diet and exercise alone. HMG-CoA reductase inhibitors (statins) are useful for treating conditions associated with high LDL levels. Other important anti-lipidemia drugs include fibrates such as gemfibril and clofibrate, bile acid sequestrant such as cholestyramine and colestipol, probucol, and nicotinic acid analogs.

Elevated cholesterol levels are in turn associated with a number of disease states, including coronary artery disease, angina pectoris, carotid artery disease, strokes, cerebral arteriosclerosis, and xanthoma.

Dyslipidemia, or abnormal levels of lipoproteins in blood plasma, is a frequent occurrence among diabetics, and has been shown to be one of the main contributors to the increased incidence of coronary events and deaths among diabetic subjects (see, e.g., Joslin, Ann. Chim. Med., 1927, 5: 1061-1079). Epidemiological studies since then have confirmed the association and have shown a several-fold increase in coronary deaths among diabetic subjects when compared with nondiabetic subjects (see, e.g., Garcia et al., Diabetes, 1974, 23: 105-11; and Laakso and Lehto, Diabetes Reviews, 1997, 5(4): 294-315). Several lipoprotein abnormalities have been described among diabetic subjects (Howard et al., Atherosclerosis, 1978, 30: 153-162).

In type 2 diabetes, obesity and dyslipidemia are also highly prevalent and around 70% of people with type 2 diabetes additionally have hypertension once again leading to increased mortality of cardiovascular diseases.

Hypertension, high blood pressure, occurs when smaller arteries become abnormally narrow, which causes the blood to exert excessive pressure against the vessel walls. As a consequence, the heart must work harder to maintain the blood flow against this increased resistance. Over an extended period of time, this may lead to enlargement and damage of the heart (cardiac hypertrophy). Although the body can tolerate an increase in blood pressure for months or even years, eventually, damage to blood vessels of the kidneys, the brain, and/or the eyes can occur. Hypertension may also lead to congestive heart failure.

In most hypertensives, both the systolic and diastolic pressures are raised. However, in some older people, “isolated” systolic hypertension may occur. A rise in diastolic pressure used to be considered more serious than a rise in systolic pressure, but now it is accepted that this isolated form of systolic hypertension puts affected people at considerable risk of brain damage due to stroke.

It is estimated that approximately 50 million people in the US have high blood pressure. About half of these people never know it because of the lack of specific symptoms. High blood pressure is therefore sometimes called the “silent killer.” It is further estimated that about 50 percent of all hypertensive people are women.

Of the roughly 50 million adult Americans with high blood pressure, only about 27% have their hypertension under control. Of those who have been diagnosed, about 27% are being treated with medications, but are failing to control the condition, and nearly 15% are not participating in any treatment at all.

In most cases of hypertension, the cause is unknown. This is called primary hypertension. In about 5 to 10 percent of people, high blood pressure is a secondary symptom of some other medical condition. For example, there might be an organic cause such as kidney disease, tumor of the adrenal glands, heart defects, or disorders of the nervous system.

Aggressive drug treatment of long-term high blood pressure can significantly reduce the incidence of death from heart disease and other causes in both men and women. In people with diabetes, controlling both blood pressure and blood glucose levels prevents serious complications of that disease. If patients have mild hypertension and no heart problems, then lifestyle changes may suffice to control the condition, if carried out with determination. For more severe hypertension or for mild cases that do not respond to changes in diet and lifestyle within a year, drug treatment is usually necessary. A single-drug regimen can often control mild to moderate hypertension. More severe hypertension often requires a combination of two or more drugs. Prolonged-release drugs are being developed so that they are most effective during early morning periods, when patients are at highest risk for heart attack or stroke.

There is still a need for a treatment that is useful in lowering the blood pressure and obesity related disorders.

The most popular over-the counter drugs for the treatment of obesity, phenylpropanolamine and ephedrine, and the most popular prescription drug, fenfluramine, were removed from the marketplace as a result of safety concerns. Drugs currently approved for the long-term treatment of obesity fall into two categories: (a) CNS appetite suppressants such as sibutramine and rimonabant, and (b) gut lipase inhibitors such as orlistat. CNS appetite suppressants reduce eating behavior through activation of the “satiety center” in the brain and/or by inhibition of the “hunger center” in the brain. Gut lipase inhibitors reduce the absorption of dietary fat from the gastrointestinal (GI) tract. Although appetite suppressants and gut lipase inhibitors work through very different mechanisms, they share in common the same overall goal of reducing body weight secondary to reducing the amount of calories that reach the systemic circulation. Unfortunately, these indirect therapies produce only a modest initial weight loss (approximately 5% compared to placebo) that is usually not maintained. After one or two years of treatment, most patients return to or exceed their starting weight. In addition, most approved anti-obesity therapeutics produce undesirable and often dangerous side effects that can complicate treatment and interfere with a patient's quality of life.

The lack of therapeutic effectiveness, coupled with the spiraling obesity epidemic, positions the “treatment of obesity” as one of the largest and most urgent unmet medical needs. There is, therefore, a real and continuing need for the development of improved medications that treat or prevent obesity.

The hypolipidaemic fibrates and antidiabetic thiazolidinediones separately display moderately effective triglyceride-lowering activities, although they are neither potent nor efficacious enough to be a single therapy of choice for the dyslipidaemia often observed in type 2 diabetic or metabolic syndrome patients. The thiazolidinediones also potently lower circulating glucose levels of type 2 diabetic animal models and humans. However, the fibrate class of compounds is without beneficial effects on glycaemia. Studies on the molecular actions of these compounds indicate that thiazolidinediones and fibrates exert their action by activating distinct transcription factors of the peroxisome proliferator activated receptor (PPAR) family, resulting in increased and decreased expression of specific enzymes and apolipoproteins respectively, both key-players in regulation of plasma triglyceride content.

There are several treatments currently available for treating diabetes mellitus but these treatments still remain unsatisfactory and have limitations. While physical exercise and reduction in dietary intake of calories will improve the diabetic condition, compliance with this approach can be poor because of sedentary lifestyles and excess food consumption, in particular high fat-containing food. Therefore, treatment with hypoglycemics, such as sulfonylureas (e.g., chlorpropamide, tolbutamide, tolazamide and acetohexamide) and biguanides (e.g. phenformin and metformin) are often necessary as the disease progresses. Sulfonylureas stimulate the β cells of the pancreas to secrete more insulin as the disease progresses. However, the response of the β cells eventually fails and treatment with insulin injections is necessary. In addition, both sulfonylurea treatment and insulin injection have the life threatening side effect of hypoglycemic coma, and thus patients using these treatments must carefully control dosage.

It has been well established that improved glycemic control in patients with diabetes (type I and type II) is accompanied by decreased microvasclular complications. Due to difficulty in maintaining adequate glycemic control over time in patients with type II diabetes, the use of insulin sensitizers in the therapy of type II diabetes is growing. There is also a growing body of evidence that PPARγ agonist, insulin sensitizer, may have benefits in the treatment of type II diabetes beyond their effects in improving glycemic control.

In the last decade a class of compounds known as thiazolidinediones (TZD) (e.g. U.S. Pat. Nos. 5,089,514; 4,342,771; 4,367,234; 4,340,605; and 5,306,726) have emerged as effective antidiabetic agents that have been shown to increase the sensitivity of insulin sensitive tissues, such as skeletal muscle, liver and adipose, to insulin. Increasing insulin sensitivity rather than the amount of insulin in the blood reduces the likelihood of hypoglycemic coma. Although thiazolidinediones have been shown to increase insulin sensitivity by binding to PPARγ receptors, this treatment also produces unwanted side effects such as weight gain and, for troglitazone, liver toxicity. There is a strong need to have a product able to address different health problems associated with obesity without producing side effects.

However, it has been well established that PPARs agonists are key players in establishing strategy to fight obesity. The Proliferator-Activated Receptors (PPARs) are members of the nuclear receptor superfamily that bind specific DNA response elements and in response to ligand binding, result in the activation of several genes. Three subtypes of PPARs have been cloned from the mouse and human: i.e., PPARα, PPARγ, and PPARδ. The PPARs are important regulators of carbohydrate and lipid metabolism, cell growth and differentiation, phenotype transition, apoptosis, neovascularization, immunoregulation and the inflammatory response.

Biological processes modulated by PPAR are those modulated by receptors, or receptor combinations, which are responsive to the PPAR receptor ligands. These processes include, for example, plasma lipid transport and fatty acid catabolism, regulation of insulin sensitivity and blood glucose levels, which are involved in hypoglycemia/hyperinsulinemia (resulting from, for example, abnormal pancreatic β cell function, insulin secreting tumors and/or autoimmune hypoglycemia due to autoantibodies to insulin, the insulin receptor, or autoantibodies that are stimulatory to pancreatic β cells), macrophage differentiation which lead to the formation of atherosclerotic plaques, inflammatory response, carcinogenesis, hyperplasia, and adipocyte differentiation. Certain PPARs are associated with a number of disease states including dyslipidemia, hyperlipidemia, hypercholesteremia, atherosclerosis, atherogenesis, hypertriglyceridemia, heart failure, myocardial infarction, vascular diseases, cardiovascular diseases, hypertension, obesity, inflammation, arthritis, cancer, Alzheimer's disease, skin disorders, respiratory diseases, ophthalmic disorders, IBDs (irritable bowel disease), ulcerative colitis and Crohn's disease. Accordingly, molecules that modulate the activity of PPARs are useful as therapeutic agents in the treatment of such diseases.

Subtypes of PPAR include PPARα, PPARδ (also known as NUC1, PPARβ and FAAR) and two isoforms of PPARγ. These PPARs can regulate expression of target genes by binding to DNA sequence elements, termed PPAR response elements (PPRE). To date, PPRE's have been identified in the enhancers of a number of genes encoding proteins that regulate lipid metabolism suggesting that PPARs play a pivotal role in the adipogenic signaling cascade and lipid homeostasis (Keller and Wahli, Trends Endoodn. Met., 1993, 4: 291-296).

PPARα is the main subtype in the liver and has facilitated analysis of the mechanism by which peroxisome proliferators exert their pleiotropic effects. PPARα is activated by a number of medium and long-chain fatty acids, and it is involved in stimulating β-oxidation of fatty acids. PPARα is also involved with the activity of fibrates and fatty acids in rodents and humans. Fibric acid derivatives such as clofibrate, fenofibrate, bezafibrate, ciprofibrate, beclofibrate and etofibrate, as well as gemfibrozil, produce a substantial reduction in plasma triglycerides along with moderate reduction in low-density lipoprotein (LDL) cholesterol, and they are used particularly for the treatment of hypertriglyceridemia.

PPARγ is the main subtype in adipose tissue and involved in activating the program of adipocyte differentiation. PPARγ is not involved in stimulating peroxisome proliferation in the liver. There are two isomers of PPARγ: PPARγ1 and PPARγ2, which differ only in that PPARγ2 contains an additional 28 amino acids present at the amino terminus. The DNA sequences for the PPARγ receptors are described in Elbrecht et al. (BBRC, 1996, 224: 431-437). Although peroxisome proliferators, including the fibrates and fatty acids, activate the transcriptional activity of PPAR's, only prostaglandin J₂ derivatives have been identified as natural ligands for PPARγ, which also binds the anti-diabetic agents thiazolidinediones with high affinity. The physiological functions of PPARα and PPARγ in lipid and carbohydrate metabolism were uncovered once it was recognized that they were the receptors for the fibrate and glitazone drugs, respectively.

In addition to the presence of a ligand, the activity of PPARγ has been shown to be influenced by the presence of coactivators and corepressors. When co-expressed in cells alongside PPARγ these proteins have been shown to greatly increase or repress the transcriptional activity of PPARγ. Differences in expression of these coactivators and corepressors between cell types may explain the observed differences in PPARγ mediated transcriptional activity between cells from different tissues.

One such coactivator is PGC-1 (Puigserver et al., Cell, 1998, 92: 829-839). The expression of this 90 kDa nuclear protein is greatly increased in muscle and brown fat of mice upon their exposure to cold temperatures. Co-expression of PGC-1 with PPARγ has been shown to activate aspects of the adaptive thermogenic program.

Activators of the nuclear receptor PPAR-γ (or alternatively, PPARγ), for example troglitazone, have been clinically shown to enhance insulin-action, to reduce serum glucose and to have small but significant effects on reducing serum triglyceride levels in patients with type 2 diabetes (see, for example, Kelly et al., Curr. Opin. Endocrinol. Diabetes, 1998, 5: 90-96, 5; Johnson et al., Ann. Pharmacother., 1997, 32:337-348; Leutenegger et al., Curr. Ther. Res., 1997, 58: 403-416).

The third subtype of PPAR, PPAR-δ (or alternatively, PPARδ, PPARβ, or NUC1) initially received much less attention than the other PPARs because of its ubiquitous expression and the unavailability of selective ligands. However, genetic studies and recently developed synthetic PPAR-δ agonists have helped reveal its role as a powerful regulator of fatty acid catabolism and energy homeostasis. Studies in adipose tissue and muscle have begun to uncover the metabolic functions of PPAR-δ. Transgenic expression of an activated form of PPAR-δ in adipose tissue produces lean mice that are resistant to obesity, hyperlipidemia and tissue steatosis induced genetically or by a high-fat diet. The activated receptor induces genes required for fatty acid catabolism and adaptive thermogenesis. Interestingly, the transcription of PPAR-γ target genes for lipid storage and lipogenesis remain unchanged. In parallel, PPAR-δ-deficient mice challenged with a high-fat diet show reduced energy uncoupling and are prone to obesity. Together, these data identify PPAR-δ as a key regulator of fat-burning, a role that opposes the fat-storing function of PPAR-γ. Thus, despite their close evolutionary and structural kinship, PPAR-γ and PPAR-δ regulate distinct genetic networks. In skeletal muscle, PPAR-δ likewise upregulates fatty acid oxidation and energy expenditure, to a far greater extent than does the lesser-expressed PPAR-α (Evans et al., Nature Med, 2004, 10(4): 1-7).

PPARδ is broadly expressed in the body and has been shown to be a valuable molecular target for treatment of dyslipidemia and other diseases. For example, in a recent study in insulin-resistant obese rhesus monkeys, a potent and selective PPARδ compound was shown to decrease VLDL and increase HDL in a dose response manner (Oliver et al., Proc. Natl. Acad. Sci. U.S.A., 2001, 98: 5305). Also, in a recent study in wild-type and HDL-lacking, ABCA1.sup.−/− mice, a different potent and selective PPARδ compound was shown to reduce fractional cholesterol absorption in the intestine, and coincidentally reduce expression of the cholesterol-absorption protein NPC1L1 (van der Veen et al., J. Lipid Res., 2005 46: 526-534).

Because of PPARs have been shown to play important roles in energy homeostasis and other important biological processes in human body and have been shown to be important molecular targets for treatment of metabolic and other diseases (see Wilson, et al., J. Med. Chem., 2000, 43: 527-550), it is desired to identify new products which are capable of interacting with PPARs without side effect to improve obesity related disorders. Such products would find a wide variety of uses, such as, for example, in the treatment or prevention of obesity, for the treatment or prevention of diabetes, dyslipidemia, metabolic syndrome X and other uses.

### SUMMARY OF THE INVENTION

It is provided herein a method of modulating a peroxisome proliferator activated receptor (PPAR) activity in a patient comprising administering to said subject an effective amount of a malleable protein matrix (MPM).

It is intended herein that the present MPM consist essentially of an isolated fermented whey protein formulation comprising at least one lactic acid bacteria microorganism; a concentrated agglomerate of whey proteins; and a peptide resulting from the hydrolysis of the whey proteins.

In another embodiment, the present MPMs are administered in combination with one or more further pharmacologically active substances selected from antiobesity agents, appetite regulating agents, antidiabetics, antihypertensive agents, agents for the treatment of complications resulting from or associated with diabetes, and agents for the treatment of complications and disorders resulting from or associated with obesity.

Suitable additional substances may be selected from CART (cocaine amphetamine regulated transcript) agonists, NPY (neuropeptide Y) antagonists, MC4 (melanocortin 4) agonists, orexin antagonists, TNF (tumor necrosis factor) agonists, CRF (corticotropin releasing factor) agonists, CRF BP (corticotropin releasing factor binding protein) antagonists, urocortin agonists, .beta.3 agonists, MSH (melanocyte-stimulating hormone) agonists, MCH (melanocyte-concentrating hormone) antagonists, CCK (cholecystokinin) agonists, serotonin re-uptake inhibitors, serotonin and noradrenaline re-uptake inhibitors, mixed serotonin and noradrenergic compounds, 5HT (serotonin) agonists, bombesin agonists, galanin antagonists, growth hormone, growth hormone releasing compounds, TRH (thyreotropin releasing hormone) agonists, UCP 2 or 3 (uncoupling protein 2 or 3) modulators, leptin agonists, dopamine (DA) agonists (bromocriptin, doprexin), lipase/amylase inhibitors, RXR (retinoid X receptor) modulators or TREβ agonists.

Suitable antiobesity agents include phentermine, leptin, bromocriptine, dexamphetamine, amphetamine, fenfluramine, dexfenfluramine, sibutramine, orlistat, dexfenfluramine, mazindol, phentermine, phendimetrazine, diethylpropion, fluoxetine, bupropion, topiramate, diethylpropion, benzphetamine, phenylpropanolamine or ecopipam, ephedrine, pseudoephedrine or cannabinoid receptor antagonists;

Suitable antidiabetics include insulin, orally active hypoglycaemic agents, and GLP-1 (glucagon like peptide-1) derivatives (see International application publication no. WO 98/08871).

Orally active hypoglycaemic agents preferably include sulphonylureas, biguanides, meglitinides, glucosidase inhibitors, glucagon antagonists (see International application publication no. WO99/01423), GLP-1 agonists, potassium channel openers (see International application publication nos. WO 97/26265 and WO 99/03861), DPP-IV (dipeptidyl peptidase-IV) inhibitors, inhibitors of hepatic enzymes involved in stimulation of gluconeogenesis and/or glycogenolysis, glucose uptake modulators, compounds modifying the lipid metabolism (e.g., antihyperlipidemic agents and antilipidemic agents), compounds lowering food intake, RXR agonists, agents acting on the ATP-dependent potassium channel of the β-cells, and thiazolidinediones (e.g., troglitazone, ciglitazone, pioglitazone and rosiglitazone).

Agents to be administered in combination with compounds of the present invention also include sulphonylureas (e.g., tolbutamide, glibenclamide, glipizide, and glicazide), biguanides (e.g., metformin), meglitinides (e.g., repaglinide and senaglinide), .alpha.-glucosidase inhibitors (e.g., miglitol and acarbose), an agent acting on the ATP-dependent potassium channel of the .beta.-cells (e.g., the above sulphonylureas and repaglinide), and nateglinide.

Antihyperlipidemic or antilipidemic agents include apolipoprotein A-I Milano, cholestyramine, colestipol, clofibrate, gemfibrozil, fenofibrate, bezafibrate, tesaglitazar, muraglitazar, EML-4156, LY-518674, LY-519818, MK-767, torcetrapib, atorvastatin, fluvastatin, lovastatin, pravastatin, simvastatin, cerivastin, rosuvastatin, pitavastatin, acipimox, ezetimibe, probucol, dextrothyroxine and nicotinic acid.

In another embodiment the present compounds are administered in combination with more than one of the above-mentioned compounds (e.g, in combination with a sulphonylurea and metformin, a sulphonylurea and acarbose, repaglinide and metformin, insulin and a sulphonylurea, insulin and metformin, or insulin and lovastatin).

Examples of antihypertensive agents include loop diuretics such as ethacrynic acid, furosemide and torsemide; diuretics such as thiazide derivatives, chlorithiazide, hydrochlorothiazide, amiloride; angiotensin converting enzyme (ACE) inhibitors such as benazepril, captopril, enalapril, fosinopril, lisinopril, moexipril, perinodopril, quinapril, ramipril and trandolapril; inhibitors of the Na-K-ATPase membrane pump such as digoxin; neutralendopeptidase (NEP) inhibitors e.g. thiorphan, terteo-thiorphan, SQ29072; ECE inhibitors e.g. SLV306; ACE/NEP inhibitors such as omapatrilat, sampatrilat and fasidotril; angiotensin II antagonists such as candesartan, eprosartan, irbesartan, losartan, telmisartan and valsartan, in particular valsartan; renin inhibitors such as aliskiren, terlakiren, ditekiren, RO 66-1132, RO-66-1168; .beta.-adrenergic receptor blockers such as acebutolol, atenolol, betaxolol, bisoprolol, metoprolol, nadolol, propranolol, sotalol and timolol; inotropic agents such as digoxin, dobutamine and milrinone; calcium channel blockers such as amlodipine, bepridil, diltiazem, felodipine, nicardipine, nimodipine, nifedipine, nisoldipine and verapamil; aldosterone receptor antagonists; and aldosterone synthase inhibitors; In another embodiment the present compounds are administered in combination with:

a) a HDL increasing compound;

b) Cholesterol absorption modulator such as Zetia™ and KT6-971;

c) Apo-A1 analogues and mimetics;

d) thrombin inhibitors such as Ximelagatran;

e) aldosterone inhibitors such as anastrazole, fadrazole, eplerenone;

f) Inhibitors of platelet aggregation such as aspirin, clopidogrel bisulfate;

g) estrogen, testosterone, a selective estrogen receptor modulator, a selective androgen receptor modulator;

or, in each case a pharmaceutically acceptable salt thereof; and optionally a pharmaceutically acceptable carrier.

In a preferred embodiment, it is provided a method of modulating peroxisome proliferator activated receptor (PPAR) activity in a patient comprising administering to the subject an effective amount of a malleable protein matrix (MPM) or formulation described herein.

In a preferred embodiment, MPM is a whey proteins hydrolysate as defined by Chemical Abstract Service No. 308074-13-7.

More particularly, PPAR activity is at least one of PPARα activity, PPARδ activity or PPARγ activity, or any combination thereof.

The modulation of PPAR activity can increase the amount of free fatty acids in the patient, stabilize blood lipids and glucose levels in the patient, reduce plasma triglycerides levels in the patient, and/or reduce cholesterols levels in the patient.

The MPM described herein can be administered concurrently with another therapeutic agent.

It is also provided the use of a malleable protein matrix (MPM) for modulating a peroxisome proliferator activated receptor (PPAR) activity in a patient.

Further provided is the use of a malleable protein matrix (MPM) in the manufacture of a medicament for modulating a peroxisome proliferator activated receptor (PPAR) activity in a patient.

Also provided herein is a pharmaceutical composition for modulating a peroxisome proliferator activated receptor (PPAR) activity comprising a therapeutically effective amount of a malleable protein matrix (MPM) and a pharmaceutically acceptable excipient.

The composition described herein can be formulated as a medicament, a dietary supplement, a nutraceutical or a functional food.

It is also encompass the use of MPM to treat or prophylaxis of a disease related to PPAR modulation or dysfunction.

It is encompass that the modulation of PPAR activity improves at least one of a pathology or symptomology of a disease or a condition selected from the group consisting of dyslipidemia, hyperlipidemia, hypercholesteremia, atherosclerosis, atherogenesis, hypertriglyceridemia, heart failure, myocardial infarction, vascular diseases, cardiovascular diseases, hypertension, obesity, cachexia, inflammation, arthritis, cancer, anorexia, anorexia nervosa, bulimia, Alzheimer's disease, skin disorders, respiratory diseases, ophthalmic disorders, irritable bowel diseases, ulcerative colitis, Crohn's disease, type-1 diabetes, type-2 diabetes and Syndrome X.

The disease can also be selected from the group consisting of HIV wasting syndrome, long term critical illness, decreased muscle mass, decreased muscle strength, decreased lean body mass, maintenance of muscle strength, maintenance of function in elderly patient, diminished muscle endurance, diminished muscle function and frailty in elderly patient.

The disease or condition can be dyslipidemia, hyperlipidemia, hypercholesteremia, atherosclerosis, atherogenesis, hypertriglyceridemia, heart failure, myocardial infarction, vascular diseases, cardiovascular diseases, hypertension, obesity, cachexia, inflammation, arthritis, cancer, anorexia, anorexia nervosa, bulimia, Alzheimer's disease, skin disorders, respiratory diseases, ophthalmic disorders, irritable bowel diseases, ulcerative colitis, Crohn's disease, type-1 diabetes, type-2 diabetes, Syndrome X, or HIV wasting syndrome, long term critical illness, decreased muscle mass, decreased muscle strength, decreased lean body mass, maintenance of muscle strength, maintenance of function in elderly patient, diminished muscle endurance, diminished muscle function or frailty in elderly patient.

It should be understood that any suitable combination of the MPM according to the invention with one or more of the above-mentioned compounds and optionally one or more further pharmacologically active substances are considered to be within the scope of the present invention.

### BRIEF DESCRIPTION OF THE DRAWINGS

Reference will now be made to the accompanying drawings:

FIG. 1A illustrates the effect of MPM on body weight gain of mice on a high carbohydrate diet;

FIG. 1B illustrates the effect of MPM on epididymal fat pads weight of mice on a high carbohydrate diet;

FIG. 1C illustrates the effect of MPM on body composition;

FIG. 2A illustrates the effect of MPM on total plasma triglycerides level in a poloxamer-induced hyperlipidemia rat model;

FIG. 2B illustrates the effect of MPM on total plasma cholesterol level in a poloxamer-induced hyperlipidemia rat model;

FIG. 2C illustrates the effect of MPM on the reduction percentage of triglycerides and cholesterol level in total plasma in a poloxamer-induced hyperlipidemia rat model;

FIG. 3A illustrates the effect of MPM on the fasting blood glucose tolerance test (OGTT) of rats on a high fructose diet;

FIG. 3B illustrates the effect of MPM on the plasma glucose area under the curve (AUC) of rats on a high fructose diet;

FIG. 3C illustrates the effect of an alternated treatment of MPM (30 days) or Water (30 days) on the plasma glucose area under the curve (AUC) of rats on a high fructose diet;

FIG. 4 illustrates the effect of MPM on systolic blood pressure (SBP) of spontaneously hypertensive rats (SHR);

FIG. 5 illustrates genes with increased and decreased expression after Weight loss in Epididymal fat (WAT).

FIG. 6 illustrates genes with increased and decreased expression after Weight loss in the liver;

FIG. 7 illustrates the effect of MPM on the reduction percentage of cholesterol level in total plasma in a randomized, double-blinded, placebo-controlled clinical trial on hypercholesterolemia;

FIG. 8 illustrates the effect of MPM on the reduction percentage of triglycerides level in total plasma in a randomized, double-blinded, placebo-controlled clinical trial on hypercholesterolemia;

FIG. 9 illustrates the effect of MPM on the reduction percentage of triglycerides level in total plasma in a randomized, double-blinded, placebo-controlled clinical trial on metabolic syndrome; and

FIG. 10 illustrates the effect of MPM on the reduction percentage of fasting glucose level in total plasma in a randomized, double-blinded, placebo-controlled clinical trial on metabolic syndrome.

### DETAILED DESCRIPTION

In accordance with the present disclosure, there is provided a malleable protein matrix (MPM) generated from the fermentation of whey by lactic acid bacteria that modulates the peroxisome proliferator activated receptors (PPARs) and some biological pathway associated to obesity and/or its symptomology, which invention comprises administering to the animal a therapeutically effective amount of MPM.

The present invention is based on the observation that modulation of the activity of PPARs and, as such, are useful for treating diseases or disorders in which PPARs contributes to the pathology and/or symptomology of the disease. This invention further provides MPM for use in the preparation of medicaments, dietary supplement, nutraceutical, functional food or food, for the treatment of diseases or disorders in which PPARs contributes to the pathology and/or symptomology of the disease.

Such MPM may therefore be employed for the treatment of prophylaxis, dyslipidemia, hyperlipidemia, hypercholesteremia, atherosclerosis, atherogenesis, hypertriglyceridemia, heart failure, hypercholesteremia, myocardial infarction, vascular diseases, cardiovascular diseases, hypertension, obesity, cachexia, HIV wasting syndrome, inflammation, arthritis, cancer, Alzheimer's disease, anorexia, anorexia nervosa, bulimia, skin disorders, respiratory diseases, ophthalmic disorders, IBDs (irritable bowel disease), ulcerative colitis and Crohn's disease; preferably for the treatment of prophylaxis, dyslipidemia, hyperlipidemia, hypercholesteremia, atherosclerosis, atherogenesis, hypertriglyceridemia, cardiovascular diseases, hypertension, obesity, inflammation, cancer, skin disorders, IBDs (irritable bowel disease), ulcerative colitis and Crohn's disease.

MPMs can also be employed to treat long term critical illness, increase muscle mass and/or muscle strength, increase lean body mass, maintain muscle strength and function in the elderly, enhance muscle endurance and muscle function, and reverse or prevent frailty in the elderly.

Further, the MPMs may be employed in mammals as hypoglycemic agents for the treatment and prevention of conditions in which impaired glucose tolerance, hyperglycemia and insulin resistance are implicated, such as type-1 and type-2 diabetes, Impaired Glucose Metabolism (IGM), Impaired Glucose Tolerance (IGT), Impaired Fasting Glucose (IFG), and Syndrome X. Preferably type-1 and type-2 diabetes, Impaired Glucose Metabolism (IGM), Impaired Glucose Tolerance (IGT) and Impaired Fasting Glucose (IFG).

In accordance with the foregoing, it is further provided a method for preventing or treating any of the diseases or disorders described above in a subject in need of such treatment, which method comprises administering to said subject a therapeutically effective amount of MPMs or a pharmaceutically acceptable formulation. For any of the above uses, the required dosage will vary disclosure also concerns: i) a MPM or an acceptable formulation for use as a dietary supplement, nutraceutical, functional food, medical food or medicament and ii) the use of a MPM or a pharmaceutically acceptable formulation for the manufacture of a medicament, dietary supplement, nutraceutical, functional food or medical food for preventing or treating any of the diseases, disorders or symptoms described above.

The present disclosure is particularly useful for preventing or treating any obesity associated disorders and the weight related body homeostasis.

Obviously, adipose tissue plays an important role in controlling whole-body homeostasis as the regulatory tissue modulating glucose and lipid homeostasis in humans. Moreover, PPARs has a profound effect on adipocytes activity. In the lean state, small adipocytes efficiently store fatty acids as triglyceride (TG). In this condition, the insulin-stimulated glucose uptake is normal. Excess caloric intake leads to metabolic overload, increased TG input and adipocyte enlargement. When overloading with TG, hypertrophy of adipocytes and increased secretion of macrophage chemoattractants occurs resulting in additional macrophages in the adipose tissue. This recruitment in turn results in a pro-inflammatory state in obese adipose tissue. Infiltrating macrophages secrete large amounts of tumor necrosis factor-α (TNF-α), which results in a chronic inflammation state with impaired TG deposition and increased lipolysis. The excess of circulating TG and free fatty acids results in the accumulation of activated lipids in the muscle, disrupting functions such as insulin-stimulated glucose transport, leading to insulin resistance and type 2 diabetes, and ectopic storage of lipid within liver, and other non-adipose tissues.

These biological processes associated with obesity cause adiposity dysfunctions. Dysfunctions in adipose tissue metabolism have a direct impact on lipid and glucose homeostasis. Adipose dysfunctions in obesity include secretions of abnormal levels of cytokines linked to insulin resistance, impairments in triglyceride storage and increases in lipolysis. These abnormalities in turn can contribute to increased fatty acids in the circulation and lead to an overload of fatty acids in the skeletal muscle and the liver.

Obviously, the plasma lipid transport, fatty acid catabolism and inflammatory response are some of the biological processes modulated by PPAR. This modulation can prevent or treat obesity, insulin resistance, type 2 diabetes, fatty liver disease, hyperlipidemia, dyslipidemia, metabolic syndrome and cardiovascular disease.

Plasma free fatty acid levels are elevated in obesity. Free fatty acid accumulation in liver produces low-grade inflammation through the activation of the nuclear factor-kappaB (NF-κB) mediated by the TLR-4 pathway. This pathway is essential for the development of innate immunity to pathogens. The liver is sensing the excess of nutriments (free fatty acids) like infectious pathogens and uses the same signaling pathway (TLR-4) resulting in the release of several proinflammatory (TNF-α, IL-1b, IL-6) and proatherogenic cytokines (MCP-1). Thus, elevated free fatty acid levels (due to obesity or to high-fat feeding) cause insulin resistance in liver, which contributes to the development of type 2 diabetes, and produce low-grade inflammation, which contributes to the development of atherosclerotic vascular diseases, nonalcoholic fatty liver disease and metabolic syndrome. Modulating the free fatty acids-related release by the modulation of PPARs in the liver would prevent those diseases.

A whey-derived ingredient, the malleable protein matrix (MPM), is produced from fermented residual whey obtained from the cheese industry (see International application publication no. WO 03/053158, the entire content of which is hereby incorporated by reference). The MPM is obtained by triggering agglomeration of whey proteins, which are then retrieved by various means. Following the agglomeration, the resulting matrix is retrieved by filtration, centrifugation or with any other methods allowing such retrieval. The protein agglomeration can be triggered by, but not limited to, a modulation of pH, temperature, the addition of salts, the addition of proteolytic enzymes, the addition of flocculent or the combination of all or some of those methods.

The malleable protein matrix (MPM) is produced with the help of a fermentation process of whey using lactic acid bacteria (LAB). It has the appearance of a malleable gel cream-coloured and of neutral odour and taste. The product has been classified as a whey proteins hydrolysate by the Chemical Abstract Service (CAS) and given the number 308074-13-7. Essentially, MPM consist of an isolated fermented whey protein formulation comprising at least one lactic acid bacteria microorganism, a concentrated agglomerate of whey proteins, and a peptide resulting from the hydrolysis of the whey proteins.

The preferred microorganism used in the fermentation process of whey is a pure strain of lactobacillus isolated from a consortium obtained from Kefir grain, R2C2 (Accession Number 041202-3, National Microbiology Laboratory, Health Canada, 1015 Arlington Street, Winnipeg, Manitoba, Canada, R3E 3R2; WO 03/053158). The process can integrate a variety of other microorganisms either alone or in combination, selected from the group consisting of Bifidobacterium adolescentis, Bifidobacterium angulatum, Bifidobacterium animalis, Bifidobacterium asteroides, Bifidobacterium bifidum, Bifidobacterium bourn, Bifidobacterium breve, Bifidobacterium catenulatum, Bifidobacterium choerinum, Bifidobacterium coryneforme, Bifidobacterium cuniculi, Bifidobacterium dentium, Bifidobacterium gallicum, Bifidobacterium gallinarum, Bifidobacterium indicum, Bifidobacterium infantis, Bifidobacterium longum, Bifidobacterium longum DJ010A, Bifidobacterium longum NCC2705, Bifidobacterium magnum, Bifidobacterium merycicum, Bifidobacterium minimum, Bifidobacterium pseudocatenulatum, Bifidobacterium pseudolongum, Bifidobacterium pseudolongum subsp. globosum, Bifidobacterium pullorum, Bifidobacterium ruminantium, Bifidobacterium saeculare, Bifidobacterium scardovii, Bifidobacterium subtile, Bifidobacterium suis, Bifidobacterium thermacidophilum, Bifidobacterium thermacidophilum subsp. suis, Bifidobacterium thermophilum, Bifidobacterium urinalis, Lactobacillus acetotolerans, Lactobacillus acidipiscis, Lactobacillus acidophilus, Lactobacillus agilis, Lactobacillus algidus, Lactobacillus alimentarius, Lactobacillus amylolyticus, Lactobacillus amylophilus, Lactobacillus amylovorus, Lactobacillus animalis, Lactobacillus arizonensis, Lactobacillus aviarius, Lactobacillus bifermentans, Lactobacillus brevis, Lactobacillus buchneri, Lactobacillus casei, Lactobacillus cellobiosus, Lactobacillus coleohominis, Lactobacillus collinoides, Lactobacillus coryniformis, Lactobacillus coryniformis subsp. coryniformis, Lactobacillus coryniformis subsp. torquens, Lactobacillus crispatus, Lactobacillus curvatus, Lactobacillus cypricasei, Lactobacillus delbrueckii, Lactobacillus delbrueckii subsp. bulgaricus, Lactobacillus delbrueckii subsp. delbrueckii, Lactobacillus delbrueckii subsp. lactis, Lactobacillus durianis, Lactobacillus equi, Lactobacillus farciminis, Lactobacillus ferintoshensis, Lactobacillus fermentum, Lactobacillus fomicalis, Lactobacillus fructivorans, Lactobacillus frumenti, Lactobacillus fuchuensis, Lactobacillus gallinarum, Lactobacillus gasseri, Lactobacillus graminis, Lactobacillus hamsteri, Lactobacillus helveticus, Lactobacillus helveticus subsp. jugurti, Lactobacillus heterohiochii, Lactobacillus hilgardii, Lactobacillus homohiochii, Lactobacillus intestinalis, Lactobacillus japonicus, Lactobacillus jensenii, Lactobacillus johnsonii, Lactobacillus kefir, Lactobacillus kefiri, Lactobacillus kefuranofaciens, Lactobacillus kefirgranum, Lactobacillus kimchii, Lactobacillus kunkeei, Lactobacillus leichmannii, Lactobacillus letivazi, Lactobacillus lindneri, Lactobacillus malefermentans, Lactobacillus mali, Lactobacillus maltaromicus, Lactobacillus manihotivorans, Lactobacillus mindensis, Lactobacillus mucosae, Lactobacillus murinus, Lactobacillus nagelii, Lactobacillus oris, Lactobacillus panis, Lactobacillus pantheris, Lactobacillus parabuchneri, Lactobacillus paracasei, Lactobacillus paracasei subsp. paracasei, Lactobacillus paracasei subsp. tolerans, Lactobacillus parakefiri, Lactobacillus paralimentarius, Lactobacillus paraplantarum, Lactobacillus pentosus, Lactobacillus perolens, Lactobacillus plantarum, Lactobacillus pontis, Lactobacillus psittaci, Lactobacillus reuteri, Lactobacillus rhamnosus, Lactobacillus ruminis, Lactobacillus sakei, Lactobacillus sakei L45, Lactobacillus salivarius, Lactobacillus salivarius subsp. salicinius, Lactobacillus salivarius subsp. salivarius, Lactobacillus sanfranciscensis, Lactobacillus sharpeae, Lactobacillus sp. NGR10001, Lactobacillus suebicus, Lactobacillus thermotolerans, Lactobacillus vaccinostercus, Lactobacillus vaginalis, Lactobacillus vermiforme, Lactobacillus versmoldensis, Lactobacillus zeae, Lactococcus garvieae, Lactococcus lactis, Lactococcus lactis subsp. cremoris, Lactococcus lactis subsp. hordniae, Lactococcus lactis subsp. lactis, Lactococcus lactis subsp. lactis bv. diacetylactis, Lactococcus piscium, Lactococcus plantarum, Lactococcus raffinolactis, Leuconostoc argentinum, Leuconostoc camosum, Leuconostoc citreum, Leuconostoc fallax, Leuconostoc ficulneum, Leuconostoc fructosum, Leuconostoc gasicomitatum, Leuconostoc gelidum, Leuconostoc inhae, Leuconostoc kimchii, Leuconostoc lactis, Leuconostoc mesenteroides, Leuconostoc mesenteroides subsp. cremoris, Leuconostoc mesenteroides subsp. dextranicum, Leuconostoc mesenteroides subsp. mesenteroides, Leuconostoc mesenteroides subsp. mesenteroides ATCC 8293, Leuconostoc pseudomesenteroides, Propionibacterium acidipropionici, Propionibacterium acnes, Propionibacterium australiense, Propionibacterium avidum, Propionibacterium cyclohexanicum, Propionibacterium freudenreichii, Propionibacterium freudenreichii subsp. freudenreichii, Propionibacterium freudenreichii subsp. shermanii, Propionibacterium granulosum, Propionibacterium jensenii, Propionibacterium lymphophilum, Propionibacterium microaerophilum, Propionibacterium propionicum, Propionibacterium thoenii, ES1 (Accession Number 041202-2, National Microbiology Laboratory, Health Canada, 1015 Arlington Street, Winnipeg, Manitoba, Canada, R3E 3R2, deposited Dec. 4, 2002), INIX (Accession Number 041202-4, National Microbiology Laboratory, Health Canada, 1015 Arlington Street, Winnipeg, Manitoba, Canada, R3E 3R2, deposited Dec. 4, 2002) and K2 (Accession Number 041202-1, National Microbiology Laboratory, Health Canada, 1015 Arlington Street, Winnipeg, Manitoba, Canada, R3E 3R2, deposited Dec. 4, 2002). The microorganisms are preferably homolactic but can be heterolactic.

The final product, MPM, is composed principally of fermented whey proteins and LAB. Also present in MPM are exopolysaccharides, dairy vitamins and minerals like niacin, riboflavin, calcium and a high proportion of peptides generated during the fermentation process. In animal models, a significant reduction of triglycerides, cholesterol, hypertension and weight was observed following MPM administration indicating multiple impacts on obesity related disorders (Beaulieu et al., 2009, J Med Food, 13: 509-519). All components may explain partly the effect individually, but the synergy between proteins, peptides, LAB and minerals could amplify the resulting effects.

These synergistic impacts are useful and highly wanted. Thus, based on the results of studies using animal models or human trials, it could not have been predicted that any of the existing MPMs would modulate the activity of PPARs without the genomic confirmation of this mechanism of action.

To make a good demonstration, the C57BI/6J mouse, upon feeding with a high-fat diet, may be used as a human-like model of diet-induce obesity (DIO) that closely mimics the ensuing metabolic cascade, for instance insulin resistance, leading to metabolic and pathologic complications such as the Metabolic Syndrome (MetS) and Type 2 diabetes mellitus (T2DM). This model has been used to screen a variety of drugs, nutraceuticals and natural health products for their beneficial effects on weight gain, lipid and glucose lowering properties. It has also been widely used to study gene expression modification in various organs (liver, lean muscle, white adipose tissues) in order to gain comprehension about the possible mechanism of action following either drug or nutritional treatments. This model may be also useful to study weight loss by either energy restriction procedures or by changing the fat content of the diet. In this model, MPM show an important impact in up-regulating PPARγ in WAT and accentuates the gene activation cascade observed during caloric restriction-induced weight loss. This can also be beneficial for the control of blood lipids and glucose.

MPM can be used under a humid form or dried and can be lyophilized or dried by other means and once dried, the MPMs are also compressible with a Carver press to form solid tablets. Lyophilized MPMs are compressible without the need to add any excipients to form tablets that could have multiple applications like incorporation of probiotics or drugs. MPM can integrate water, oil or other solvent to improve its general properties. MPM represent an inexpensive product with a variety of competitive advantages and applications.

Several drugs may be formulated with the MPM and they may be delivered orally and topically. A plurality of pharmaceutically related products and drugs or bioactive materials can be formulated with the MPM like small molecules of various classes (hydrophilic and hydrophobic), proteins, RNA, oligonucleotides, DNA, viruses and bacteria.

Suitable bioactive materials also include therapeutic and prophylactic agents. These include, but are not limited to any therapeutically effective biological modifier. Such modifiers include, but are not limited to lipids, organics, proteins and peptides (synthetic and natural), peptide mimetics, hormones (peptides, steroid and corticosteroid), D and L amino acid polymers, oligosaccharides, polysaccharides, nucleotides, oligonucleotides and nucleic acids, including DNA and RNA, protein nucleic acid hybrids, small molecules and physiologically active analogs thereof. Further, the modifiers may be derived from natural sources or made by recombinant or synthetic means and include analogs, agonists and homologs. As used herein “protein” refers also to peptides and polypeptides. Such proteins include, but are not limited to enzymes, biopharmaceuticals, growth hormones, growth factors, insulin, monoclonal antibodies, interferons, interleukins and cytokines.

The present disclosure encompasses a method of modulating the activity of PPARs in a human subject comprising administering to a subject, in a preventive or therapeutic approach, a malleable protein matrix in an amount effective to modulate the biological activity of PPAR.

In various embodiments, the present disclosure relates to medicaments, dietary supplements, functional food, cosmeceutical supplements and medical food.

The terms activation, stimulation and treatment, as used herein and applied to cells or to receptors, may have the same meaning, e.g., activation, stimulation, or treatment of a cell or receptor with a ligand, unless indicated otherwise by the context or explicitly. The term ligand encompasses natural and synthetic ligands, e.g., cytokines, cytokine variants, analogues, muteins, and binding compositions derived from antibodies. Also encompass are small molecules, e.g., peptide mimetics of cytokines and peptide mimetics of antibodies. The expression activation can also refer to cell activation as regulated by internal mechanisms as well as by external or environmental factors. A response, e.g., of a cell, tissue, organ, or organism, encompasses a change in biochemical or physiological behavior, e.g., concentration, density, adhesion, or migration within a biological compartment, rate of gene expression, or state of differentiation, where the change is correlated with activation, stimulation, or treatment, or with internal mechanisms such as genetic programming.

The activity of a molecule describes or refers to the binding of the molecule to a ligand or to a receptor, to catalytic activity; to the ability to stimulate gene expression or cell signaling, differentiation, or maturation; to antigenic activity, to the modulation of activities of other molecules, and the like. The activity of a molecule also refers to the activity of modulating or maintaining cell-to-cell interactions, e.g., adhesion, or the activity of maintaining a structure of a cell, e.g., cell membranes or cytoskeleton. The term activity can also mean specific activity, e.g., [catalytic activity]/[mg protein], or [immunological activity]/[mg protein]; concentration in a biological compartment, or the like. A proliferative activity encompasses an activity that promotes, that is necessary for, or that is specifically associated with, e.g., normal cell division, as well as cancer, tumors, dysplasia, cell transformation, metastasis and angiogenesis.

Administration and treatment, as it applies to an animal, human, experimental subject, cell, tissue, organ, or biological fluid, refers to contact of an exogenous pharmaceutical, therapeutic, diagnostic agent, or composition to the animal, human, subject, cell, tissue, organ, or biological fluid. Administration and treatment can refer, e.g., to therapeutic, pharmacokinetic, diagnostic, research, and experimental methods. Treatment of a cell encompasses contact of a reagent to the cell, as well as contact of a reagent to a fluid, where the fluid is in contact with the cell. Such administration and treatment also means in vitro and ex vivo treatments, e.g., of a cell, by a reagent, diagnostic, binding composition, or by another cell. A treatment, as it applies to a human, veterinary, or research subject, refers to therapeutic treatment, prophylactic or preventative measures, to research and diagnostic applications. Further, a treatment as it applies to a human, veterinary, or research subject, or cell, tissue, or organ, encompasses for example the modulation of the activity of PPAR to a human or animal subject, a cell, tissue, physiological compartment, or physiological fluid.

An effective amount, as defined herein, encompasses an amount sufficient to ameliorate or prevent a symptom or sign of the medical condition. Effective amount also means an amount sufficient to allow or facilitate diagnosis. An effective amount for a particular patient or veterinary subject may vary depending on factors such as the condition being treated, the overall health of the patient, the method route and dose of administration and the severity of side affects. An effective amount can be the maximal dose or dosing protocol that avoids significant side effects or toxic effects. The effect will result in an improvement of a diagnostic measure or parameter by at least 5%, usually by at least 10%, more usually at least 20%, most usually at least 30%, preferably at least 40%, more preferably at least 50%, most preferably at least 60%, ideally at least 70%, more ideally at least 80%, and most ideally at least 90%, where 100% is defined as the diagnostic parameter shown by a normal subject.

An important number of HIV-infected patients suffered of wasting syndrome causing an important weight loss, principally a muscle mass loss, associated with diarrhea, fever and fatigue. This wasting syndrome is an AIDS-related complication leading to an important rate of mortality. This wasting syndrome can be treated or attenuated by a modulation of PPARs and its associated pathway.

Lipodystrophy is associated with metabolic disorders such as hyperlipidemia and insulin resistance as well as an accumulation of fat in the abdomen. This complication is mostly associated with the protease inhibitors (AIDS medication) that interfere with proteolysis of transcription factors implicated in lipids homeostasis. Lipodystrophy complications lead to an adipose tissue disorder characterized by a selective loss of body fat. Patients with lipodystrophy have a tendency to develop insulin resistance, type 2 diabetes, a high blood triglyceride level and fatty liver.

Modulation of the biological activity of PPARs can be used to improve the situation of people with AIDS by the amelioration of the plasma lipid transport and fatty acid catabolism, regulation of insulin sensitivity and blood glucose levels. PPARs modulation can also improve the adiposity distribution and differentiation while reducing inflammatory of cytokines, responsible of monitoring the constant inflammatory status in HIV-patients and leading to many AIDS complications such as wasting syndrome, lipodystrohy, deregulation of immunity, and of propagating the virus. This modulation of PPARs is essential for maintaining lipids and glucose homeostasis and thus, reducing AIDS complications like hyperlipidemia and weight control.

An inhibitors”, “antagonists” or “activators” and “agonists” refer to inhibitory or activating molecules, respectively, e.g., for the activation of, e.g., a ligand, receptor, cofactor, a gene, cell, tissue or organ. A modulator of, e.g., a gene, a receptor, a ligand or a cell, is a molecule that alters an activity of the gene, receptor, ligand or cell, where the activity can be activated, inhibited or altered in its regulatory properties. The modulator may act alone or it may use a cofactor, e.g., a protein, metal ion or small molecule. Inhibitors are compounds that decrease, block, prevent, delay activation, inactivate, desensitize or down regulate, e.g., a gene, protein, ligand, receptor or cell.

An “activators” are compounds that increase, activate, facilitate, enhance activation, sensitize or up regulate, e.g., a gene, protein, ligand, receptor or cell. An “inhibitor” may also be defined as a composition that reduces, blocks or inactivates a constitutive activity. An “agonist” is a compound that interacts with a target to cause or promote an increase in the activation of the target. An “antagonist” is a compound that opposes the actions of an agonist. An antagonist prevents, reduces, inhibits or neutralizes the activity of an agonist. An antagonist can also prevent, inhibit or reduce constitutive activity of a target, e.g., a target receptor, even where there is no identified agonist.

### Pharmaceutical Compositions

To prepare pharmaceutical or sterile compositions, the MPM is admixed with a pharmaceutically acceptable carrier or excipient.

Formulations of therapeutic and diagnostic agents may be prepared by mixing with physiologically acceptable carriers, excipients, or stabilizers in the form of, e.g., lyophilized powders, slurries, aqueous solutions or suspensions.

Toxicity and therapeutic efficacy of the MPM, administered alone or in combination with an additional therapeutic agent, can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio between LD₅₀ and ED₅₀. The data obtained from these cell culture assays and animal studies can be used in formulating a range of dosage for use in human. The dosage of such compounds lies preferably within a range of circulating concentrations that include the ED₅₀ with little or no toxicity. The dosage may vary within this range depending upon the dosage form employed and the route of administration utilized.

Oral administration to the individual is preferred. Other suitable routes of administration may, for example, include rectal, cutaneous, or intestinal administration.

Determination of the appropriate dose is made by the clinician, e.g., using parameters or factors known or suspected in the art to affect treatment or predicted to affect treatment. Generally, the dose begins with an amount somewhat less than the optimum dose and it is increased by small increments thereafter until the desired or optimum effect is achieved relative to any negative side effects. Important diagnostic measures include those of symptoms of, e.g., the inflammation or level of inflammatory cytokines produced. Preferably, a biologic that will be used is derived from the same species as the animal targeted for treatment, thereby minimizing an inflammatory, autoimmune, or proliferative response to the reagent.

As used herein, the term “treat” or “treatment” includes a postponement of development of the symptoms associated with autoimmune disease or pathogen-induced immunopathology and/or a reduction in the severity of such symptoms that will or are expected to develop. The terms further include ameliorating existing uncontrolled or unwanted autoimmune-related or pathogen-induced immunopathology symptoms, preventing additional symptoms, and ameliorating or preventing the underlying causes of such symptoms. Thus, the terms denote that a beneficial result has been conferred on a vertebrate subject with an autoimmune or pathogen-induced immunopathology disease or symptom, or with the potential to develop such a disease or symptom.

As used herein, the term “therapeutically effective amount” or “effective amount” refers to an amount of MPM, that when administered alone or in combination with an additional therapeutic agent to a cell, tissue, or subject is effective to immunomodulate, prevent or ameliorate the autoimmune disease or pathogen-induced immunopathology associated disease or condition or the progression of the disease. A therapeutically effective dose further refers to that amount of the compound sufficient to result in amelioration of symptoms, e.g., treatment, healing, prevention or amelioration of the relevant medical condition, or an increase in rate of treatment, healing, prevention or amelioration of such conditions. When applied to an individual active ingredient administered alone, a therapeutically effective dose refers to that ingredient alone. When applied to a combination, a therapeutically effective dose refers to combined amounts of the active ingredients that result in the therapeutic effect, whether administered in combination, serially or simultaneously. An effective amount of therapeutic will decrease the symptoms typically by at least 10%; usually by at least 20%; preferably at least about 30%; more preferably at least 40%, and most preferably by at least 50%.

Methods for co-administration or treatment with a second therapeutic agent (concurrently or prior to/subsequent to administering the pharmaceutical composition described herein), e.g., a cytokine, steroid, chemotherapeutic agent, antibiotic, or radiation, are well known in the art. The pharmaceutical composition can also be employed with other therapeutic modalities such as phototherapy and radiation.

Typical veterinary, experimental, or research subjects include monkeys, dogs, cats, rats, mice, rabbits, guinea pigs, horses, and humans.

### Dietary Supplement, Nutraceutical/Functional or Medical Food Composition

MPM is also useful as a component of a dietary supplement, nutraceutical/functional or medical food. Dietary supplements, nutraceutical/functional or medical food suitable for use in the present invention include compositions wherein the active ingredients are contained in an effective amount to achieve its intended purpose. Determination of the effective amounts is well within the capability of those skilled in the art, especially in light of the detailed disclosure provided herein. The amount of composition administered will be dependent upon the condition being treated, the subject being treated, on the subject's weight, the severity of the affliction, the manner of administration and the judgment of the individual's physician. The ingredients of the dietary supplement of this invention are contained in acceptable excipients and/or carriers for oral consumption. The actual form of the carrier, and thus, the dietary supplement itself, may not be critical. The carrier may be a liquid, gel, gelcap, capsule, powder, solid tablet (coated or non-coated), dairy product/food product or the like. Suitable excipient and/or carriers include maltodextrin, calcium carbonate, dicalcium phosphate, tricalcium phosphate, microcrystalline cellulose, dextrose, rice flour, magnesium stearate, stearic acid, croscarmellose sodium, sodium starch glycolate, crospovidone, sucrose, vegetable gums, agar, lactose, methylcellulose, povidone, carboxymethylcellulose, corn starch, and the like (including mixtures thereof). The various ingredients and the excipient and/or carrier are mixed and formed into the desired form using conventional techniques. Dose levels/unit can be adjusted to provide the recommended levels of ingredients per day in a reasonable number of units. The dietary supplement may also contain optional ingredients including, for example, herbs, vitamins, minerals, enhancers, colorants, sweeteners, flavorants, inert ingredients, and the like. Such optional ingredients may be either naturally occurring or concentrated forms. Selection of one or several of these ingredients is a matter of formulation, design, consumer preference and end-user. The amounts of these ingredients added to the dietary supplements nutraceutical/functional or medical food of this invention are readily known to the skilled artisan.

### Cosmeceutical Composition

MPM is also useful as a component of a cosmeceutical supplement. Cosmeceutical supplements suitable for use in the present invention include compositions wherein the active ingredients are contained in an effective amount to achieve its intended purpose. Such cosmeceutical supplements can be formulated for delivery by a mode selected from the group consisting of but not restricted to oral delivery, spray, injection, drops, perfusion, irrigation, topical skin application and topical application during surgery. Determination of the effective amounts is well within the capability of those skilled in the art, especially in light of the detailed disclosure provided herein. The ingredients of the cosmeceutical supplement of this invention are contained in acceptable excipients and/or carriers for dermal application. The actual form of the carrier, and thus, the cosmeceutical product itself, may not be critical. A cosmeceutical supplement exhibiting biological properties for skin care and maintenance, repair, skin regeneration, and anti-aging in products like skin care, sunscreens, which include baby creams, emollient creams, cold creams, conditioning creams, protective creams, sunscreen lotion, lip balm, lipsticks, eye shadows and bar soaps or the like. The various ingredients and the excipient and/or carrier are mixed and formed into the desired form using conventional techniques. Dose levels/unit can be adjusted to provide the recommended levels of ingredients per day in a reasonable number of units. The cosmeceutical supplement may also contain optional ingredients including, for example, herbs, vitamins, minerals, enhancers, colorants, sweeteners, flavorants, inert ingredients and the like. Such optional ingredients may be either naturally occurring or concentrated forms. Selection of one or several of these ingredients is a matter of formulation, design, consumer preference and end-user. The amounts of these ingredients added to the cosmeceutical product of this invention are readily known to the skilled artisan.

The present invention will be more readily understood by referring to the following examples which are given to illustrate the invention rather than to limit its scope.

### Example 1

### Generation of MPM

MPM are obtained by fermenting sweet whey with lactic acid bacteria from the Lactobacillus genus, followed by a protein-specific recuperation procedure. The product is produce by Technologie Biolactis Inc. at industrial scale by fermentation of Lactobacillus Kefuranofaciens R2C2 strain like as described below.

The first step is a pre-culture in fermentor where frozen ferment culture, R2C2 (strain accession number: 041202-3; National Microbiology Laboratory, Health Canada, 1015 Arlington Street, Winnipeg, Manitoba, Canada, R3E 3R2), is used to inoculate pre-culture medium. The pre-culture medium is a medium composed of whey powder 6.7% (w/v), yeast extract 0.41% (w/v) (Biospringer, 0202), yeast peptone 0.12% (w/v) (BioSpringer, Hyp A), water 92.8%. The grow media is pasteurized at 82° C. (180° F.)+/−2° C. for 35 minutes, then cool down at 37° C. The incubation of the strain, with a ratio of initial inoculation of 10% (10⁸ bacteria/ml), is at 37° C. for 24 hours. Initial pH should be 5.3+/−0.3 and final ph after 24 h should be 3.8 (+/−0.1).

The second step is the crude cheese whey treatment in which the whey is pasteurized to destruct its microbiological flora. The cheese whey pasteurization is conducted using heat exchanger. After pasteurization, the cheese whey is inoculated with 10% (v/v) of the pre-culture. The fermentation is realized at 37° C. with controlled temperature for twelve hours. Agitation is maintained to a minimum to allow a uniform distribution but without causing an excessive aeration. The fermentation is follow-up by the addition of calcium chloride 0.3% (wt/vol) and the adjustment of the pH to 7.5 with NaOH. The fermented cheese whey is heat treated a second time using heat exchanger and the MPM recovery is achieved using a VNPX710 clarifying unit of Alpha Laval (Alfa Laval, Sweden). The consistency was adjusted to a yogurt-like by clarifying recuperation adjustment. The resulting MPM is malleable, looks like a pudding of white creamy color with no noticeable taste or smell. This MPM consists mainly (wt/wt), of water (80.3%), protein (8.6%), minerals (4.7%, of which calcium comprises 1.5%), carbohydrate (1.5%), fat (1.3%) and bacteria (6×10¹¹/100 g).

After recovery, the MPM is cooled down to 4° C. and packed in hermetic packaging. The MPM is stored at 4° C. for 5 days to complete the microbiologic analyses. They can be uses as it is or dried.

Those skilled in the art will recognize, or be able to ascertain using no more than routine experimentation, numerous equivalents to the specific procedure described herein. Such equivalents are considered to be within the scope of this invention. The international patent application publication No WO 03/053158 discloses different way of producing MPM.

### Example 2

### Effect of MPM on Weight Management and Body Composition

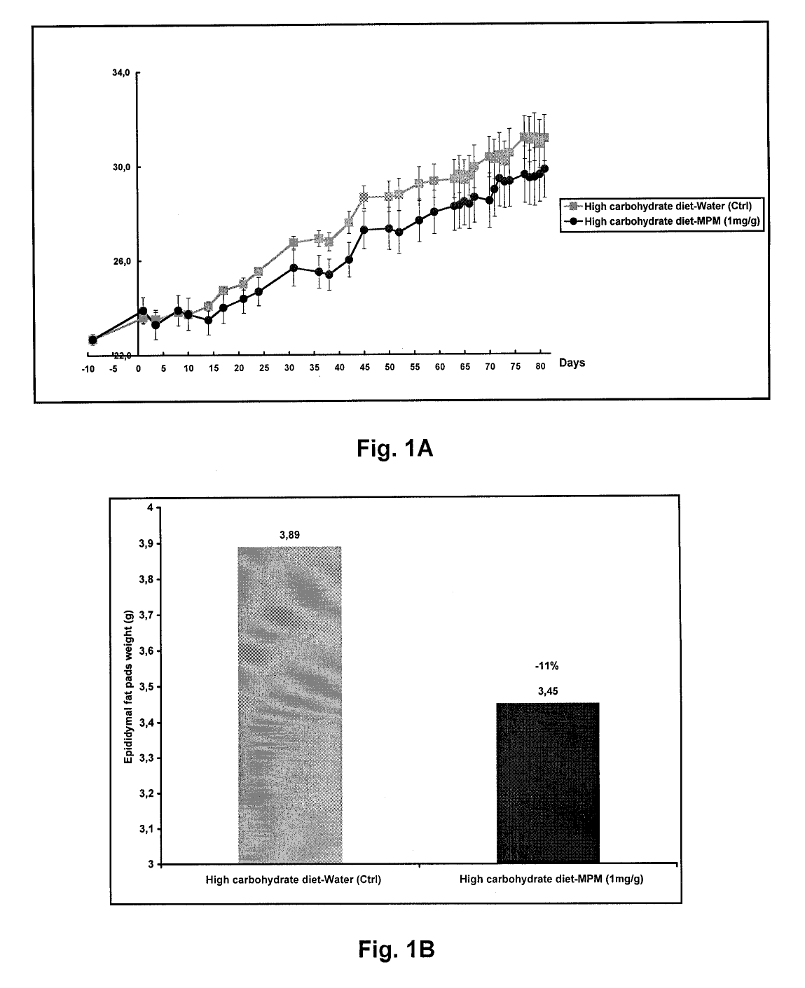

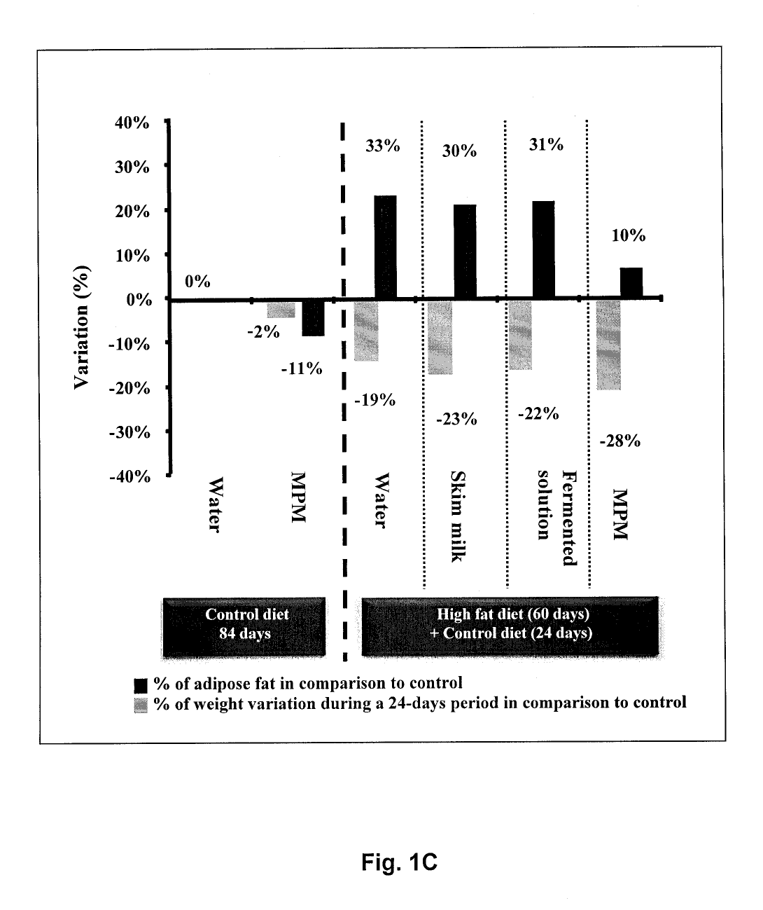

In a first experiment (FIG. 1A, 1B), male C57BI/6J mice were put on a high carbohydrate diet (48% dextrin, 19% sucrose). Treatments consisted of daily intragastric gavage (5 days a week) with water (control group, 100 μl) or MPM (1 mg/g). In the second experiment (FIG. 1C), male C57BI/6J mice were put on a high fat diet (34.7% fat) followed by a weight lost period on a control diet (5.2% fat) for 24 days. Treatments consisted of daily intragastric gavage (5 days a week) with water (control group, 100 μl), skim milk (100 μl), fermented solution (100 μl) or MPM (1 mg/g). In both experiments, animals were maintained in a 12-hour light/dark cycle and consumed their diet and water ad libitum for 12 weeks. Animal weights were recorded daily (FIG. 1A). At the end of the experiment, animals were sacrificed and epididymal fat pads weighted (FIGS. 1B and 1C).

As demonstrated, MPM has an effect on obesity and weight management. Animals fed on a high carbohydrate diet with MPM were leaner (−6%) than water-treated counter-part. It appears also that lower fat was produce in the MPM-treated group as shown in FIG. 1B (−11%). The ratio of epididymal fat weight/mice weight demonstrated that the body composition of mice differs from MPM-treated mice and water-treated mice. FIGS. 1B and 1C demonstrate that MPM reduces the fat content and modifies the body composition.

### Example 3

### Effect of MPM on Total Plasma Triglycerides and Cholesterol Level in a Poloxamer-Induced Hyperlipidemia Rat Model

Female Wistar rats were randomly assigned to various treatment groups. Animals were maintained in a 12-hour light/dark cycle and consumed standard diet and water ad libitum. Rats were pretreated by intragastric gavages daily for 7 days with water (placebo group, 1 ml), MPM (200 mg/day) or niacin (25 mg/day) which is known to exert a positive effect on triglycerides and cholesterol metabolism in this model. Following 7 days pretreatment, all animals were rendered hyperlipidemic by an i.p. injection of 300 mg of poloxamer 407 (P407, BASF Corporation). Daily intragastric gavages were continued for 3 days (72 h) during the P407-induced hyperlipidemic state. Blood samples were collected for determination of total plasma cholesterol, triglycerides at 72 h post-induction of hyperlipidemia. All blood lipid analyses were performed in an independent laboratory.

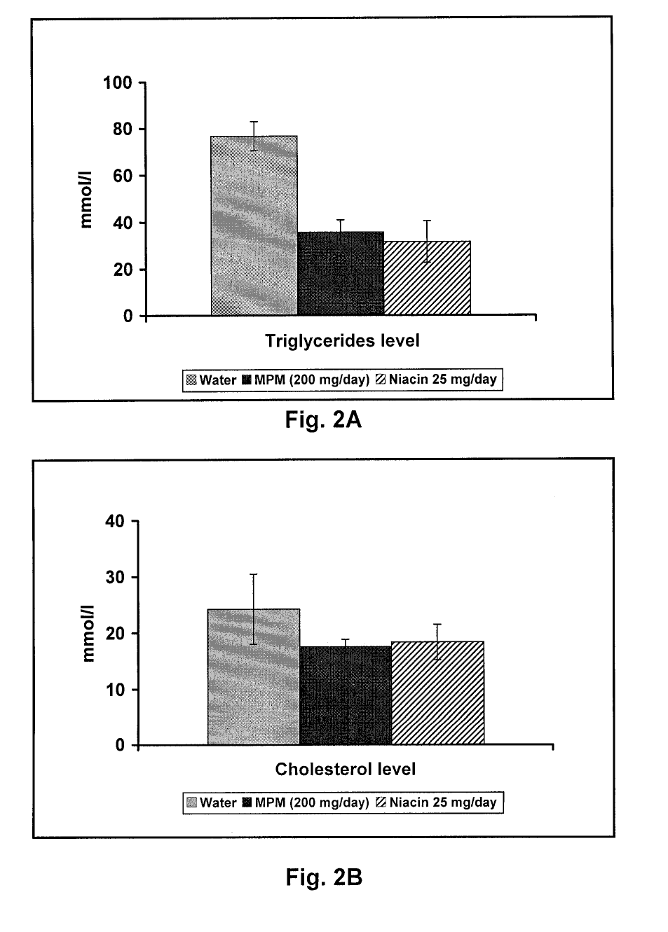

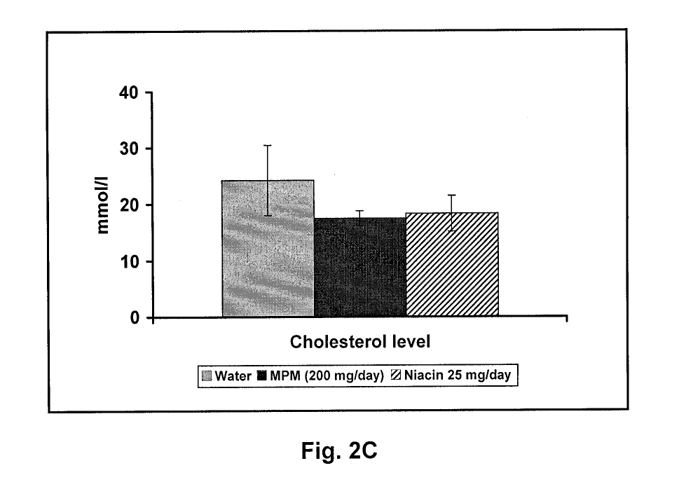

As seen in FIGS. 2A, 2B and 2C, MPM (200 mg/day) is able to reduce plasma triglycerides and cholesterol levels (−51% and −26% respectively) at 72 hours after hyperlipidemia compare to water-treated animals. These results on hyperlipidemia suggested a beneficial impact on plasma lipid levels associated with consumption of MPM. MPM showed the capacity to regulate lipid levels in laboratory animals at basal state and in the poloxamer 407-induced hyperlipidemia model. In every aspect tested, MPM seemed to have lipid-lowering properties as good as niacin. This model is also used to measure the impact on atherosclerosis.

### Example 4

### Effect of MPM on the Fasting Blood Glucose Tolerance Test (OGTT) of Rats on a High Fructose Diet

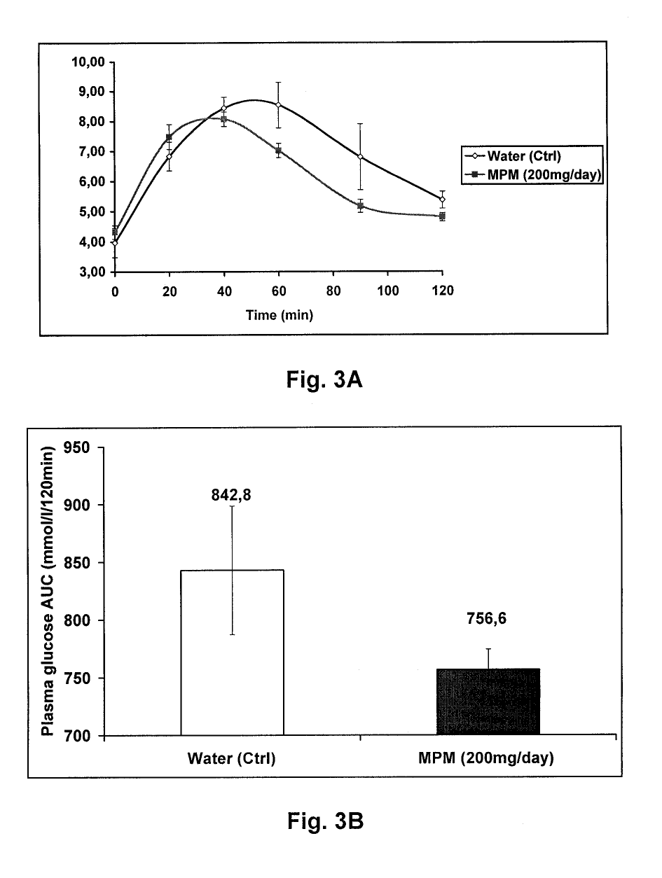

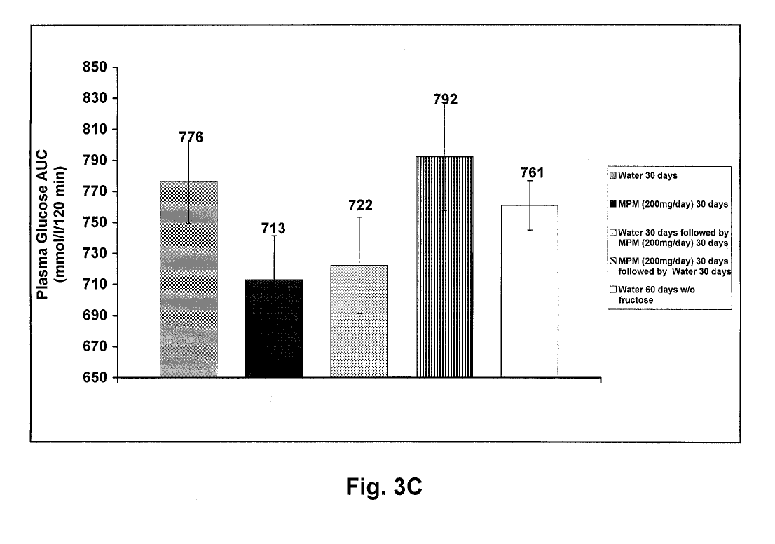

Male Wistar rats were put on a high fructose diet (10%) for two weeks. The treatments consisted of daily intragastric gavages (7 days a week) with either water (placebo group, 1 ml), or MPM (200 mg/day). Animals were maintained in a 12-hour light/dark cycle and consumed their diet and water ad libitum for 15 days. On day 15, overnight fasted animals from each group were subjected to oral glucose tolerance test (oral glucose load 2 g/kg). Thereafter, following oral glucose load, blood samples were collected at 0, 20, 40, 60, 90 and 120 min and glucose was measured using the FreeStyle™ mini apparatus from ThermaSense. The area under the curve (FIG. 3A) was then calculated to evaluate glucose sensitivity (FIG. 3B). Also, male Wistar rats were put on a high fructose diet (10%) for 30 days or normal diet (water control without fructose). The treatments consisted of daily intragastric gavages (7 days a week) with either water (1 ml), or MPM (200 mg/day). After 30 days, overnight fasted animals from each group were subjected to oral glucose tolerance test (oral glucose load 2 g/kg). Thereafter, following oral glucose load, blood samples were collected at 0, 20, 40, 60, 90 and 120 min and glucose was measured using the FreeStyle™ mini apparatus from ThermaSense. The area under the curve was then calculated to evaluate glucose sensitivity. Animals on water were then switch to MPM and vice versa (FIG. 3C).

Experiments on Wistar rats treated or not with MPM suggested that it has an impact on glucose homeostasis. After 15 days of treatment with MPM, animals on a high-fructose diet have a faster plasma glucose clearance (FIG. 3A) and a better sensitivity to insulin (FIG. 3B). This effect is reversible. Indeed, the sensitivity to insulin of animals which developed insulin resistance after one month of a high-fructose diet become normal again after one month of treatment with MPM. In contrast, stopping the administration of MPM to animals on a high-fructose diet decreases their sensitivity to insulin leading to insulin resistance (FIG. 3C).

### Example 5

### Effect of MPM on Systolic Blood Pressure (SBP) of Spontaneously Hypertensive Rats (SHR)

Spontaneously hypertensive rats (SHR, 6 weeks old) were maintained in a 12-hour light/dark cycle and consumed standard diet and water ad libitum. 10 rats were randomly assigned to various treatment groups. Rats were maintained on a regular diet for 2 weeks in order to allow the development of hypertension. Hypertensive rats were then treated for 3 weeks by daily intragastric gavages with water (placebo group, 1 ml/day), MPM (200 mg/day) or enalapril-malate (10 mg/kg; a known hypotensive agent). Daily intragastric gavages were then stopped for the last week of the experiment. Systolic blood pressure was measured weekly with the automated RTBP2000 Tail Blood Pressure system (Kent Scientific, Torrington, Conn., USA). An average of 3 measurements was taken as initial mean SBP. Data was acquired and analyzed with Biopac Student Lab Pro® software version 3.6.1 (Biopac System, USA).

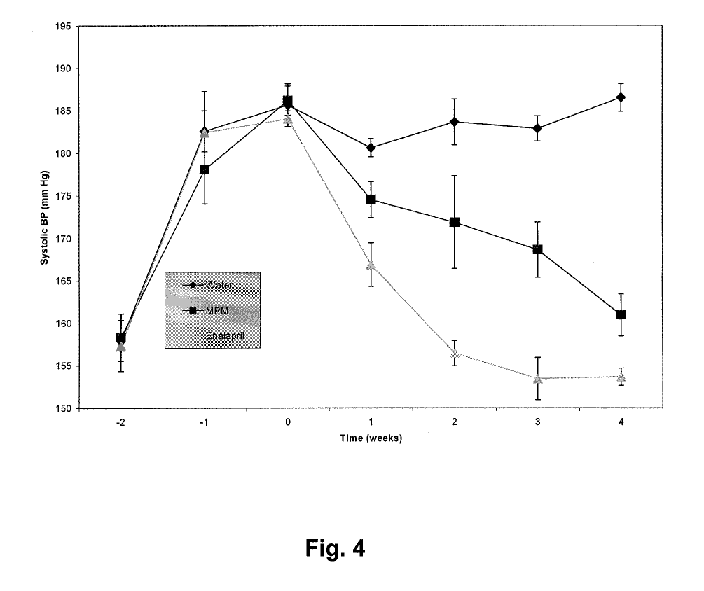

In this model, an initial period of 2 weeks was necessary to achieve high systolic blood pressure and after which treatments were started. Enalapril-treated group had a normalized SBP going from 184 mm Hg to around 153 mm Hg after 4 weeks (17% reduction) while a decrease of SBP was also observed for the MPM group with a maximum reduction of 14% at week 4 (FIG. 4) comparable to Enalapril. These results clearly show the potential of MPM for SBP reduction.

### Example 6

### Effect of MPM on Gene Expression in a Weight Management and Body Composition Study

The experiment described in Example 2 was designed to assess the impact of MPM on body weight gain during high-fat feeding of C57BI/6J and further submitted to weight loss by changing the fat content of the diet. MPM was administered by an oral gavage procedure to mimics more closely the potential human dosage and usage. Besides, gene expression modification by MPM following weight loss was evaluated in the liver and in white adipose tissue by using glucose and lipid metabolism pathway-specific arrays.

A total of 42 Male C57BI/6J mice aged between 6 and 8 weeks were purchased from Charles River (St-Constant, QC, Canada). Animals were housed 3 or 4 in a cage under specific pathogen-free conditions and maintained in a 12-hour light/dark cycle. All animals received water and food ad libitum. All procedures using mice in this study were in accordance with the institution's guide for the care and use of laboratory animals and approved by the institutional animal care and user committee of INRS-Institut-Armand-Frappier.

Following a 1-week acclimatization, weight-matching animals were divided to received either a low-fat (LF) or high-fat (HF) diet. The LF diet contained 5.2% of fat (12% of calories) and the HF diet contained 34.7% of fat (60% of calories). Each of the LF and HF group of mice were further subdivided to receive one daily intragastric gavage (5 days a week) of either water (5 ml/Kg), MPM (20% w/w, 5 ml/kg) or skim milk (20% w/w, 5 ml/kg).

Mice groups consumed their respective diet and water ad libitum for 8 weeks. After 8 weeks of diet-induced obesity (D10), the weight loss phase of HF-fed groups was initiated by switching to a LF diet while LF subgroups were kept as is for 4 more weeks. Diet and water was again provided ad libitum. Intragastric treatment gavages were maintained as described.

For each animal, 100 mg of liver or epididymal fat pads (white adipose tissue or WAT) were added separately to TRIzol™ reagent from Invitrogen (Carslbaad, Calif., USA) and left at room temperature for one hour before total RNA isolation with chloroform and precipitation with isopropanol. RNA was resuspended in RNase-free water and concentration measured on a BioRad® spectrophotometer (Hercules, Calif., USA). Total RNA (1 μg) from liver or WAT obtained at the end of the weight loss phase from Water- (n=3) or MPM-treated animals (n=3) was converted to cDNA using the RT2 PCR Array first strand kit from SuperArray™ Bioscience (Frederick, Md., USA). Gene expression was analyzed according to the manufacturer instructions (SABiosciences; Frederick, Md., USA) using the mouse lipoprotein signaling and cholesterol metabolism PAMM-080 or the mouse diabetes PAMM-023 PCR arrays. Each array contained 84 Pathway-Specific probes reflecting genes involved either in glucose and lipid metabolism.

Various array quality controls were also included as follow: 5 house keeping genes, genomic DNA contamination control, triplicate reverse transcription controls and triplicate positive PCR controls. Reactions were cycled in an Applied Biosystems ABI Prism® 7900 FAST sequence detector (Foster city, CA, USA) and acquired data were analyzed using the ΔΔCt method to determine the expression level of each transcript normalized to the expression level of housekeeping gene controls. Arrays were performed independently for each tissue for all six animals (three MPM-treated animals and three Water-treated control animals). The Ct value was used for calculations of relative amount of mRNA molecules. The Ct value of each target gene was normalized by subtraction of the Ct value from average of five housekeeping genes. Ct values for housekeeping genes were monitored for consistency between the arrays. Relative quantitative change was calculated using the formula 2-(ΔCt MPM-ΔCt untreated). The resulting values were reported as fold change. The negative controls ensured a lack of DNA contamination and set the threshold for the absent/present calls.

ANOVA using treatment as factor. Statistical difference among treatments was then evaluated using the LSD Tukey HSD multiple comparison test. Statistical difference in gene expression between the two conditions was assessed by a gene-wise, two-sample, t-test procedure. P value at 0.05 was declared significant.

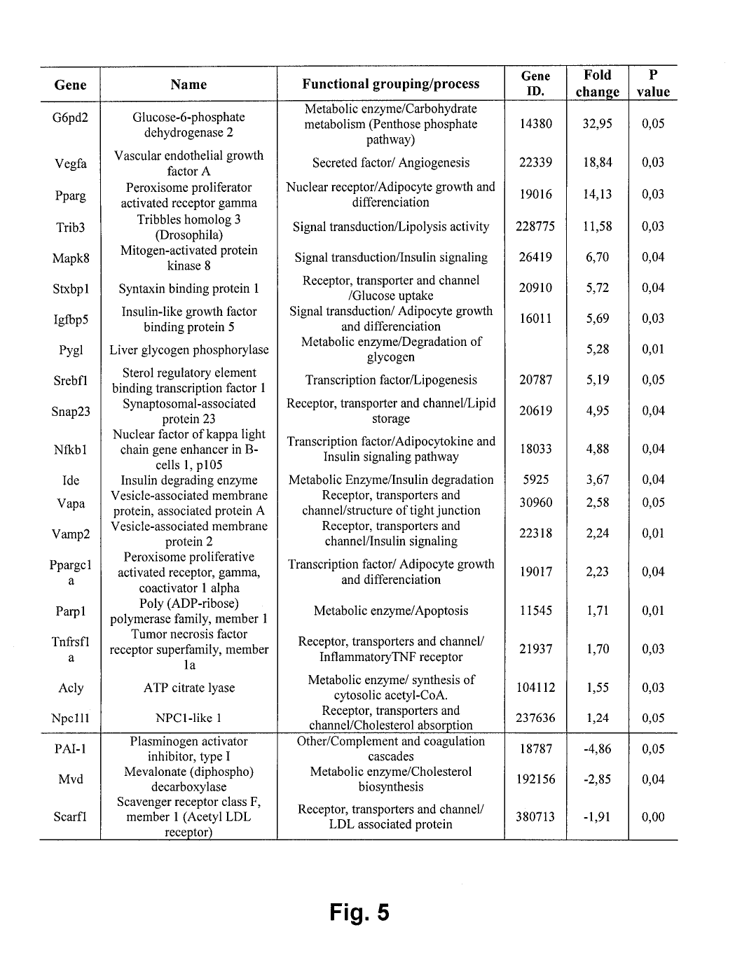

A total of 22 genes were differentially expressed in the MPM-treated WAT of C57BI/6J mice as compared to their water-treated counterpart. Of these, 19 genes were up regulated while 3 genes were down regulated (FIG. 5).

The most highly expressed gene (+33 fold) in MPM-treated mice was Glucose-6-phosphate dehydrogenase (G6pd2), a metabolic enzyme in the pentose phosphate pathway supplying NADPH which is used in many biosynthesis processes including fatty acids, cholesterol and glutahione. Among the other 19 up regulated genes, at least 7 genes are closely involved in either adipocyte growth, differenciation and/or lipid storage (Vegfa, Pparγ, Igfbp5, Srebf1, Snap23, Ppargc1a, Acly). Of note, the expression of PPARγ (Peroxisome proliferator activated receptor gamma, also identified as PPARg in FIG. 5) was highly expressed as compared to the control (+14 fold). This nuclear receptor, predominantly expressed in adipose tissue, is thought to play a key role in adipogenesis and in the ability of the adipocytes to adequately store triglycerides. The development of obesity in both genetically and diet-induced obese mice is known to repress PPARγ and many adipogenic genes although it may vary depending on the specie and time point at which measurements are made. The same pattern of reduced PPARγ is also seen in insulin resistant humans. The most highly repressed gene (−4.9 fold) in MPM-treated mice was Plasminogen activator inhibitor type I (PAI-1). Its elevation is associated with the development of obesity in High fat fed C57BI/6J mice while weight loss has been shown to reduce the plasma level of PAI-1.

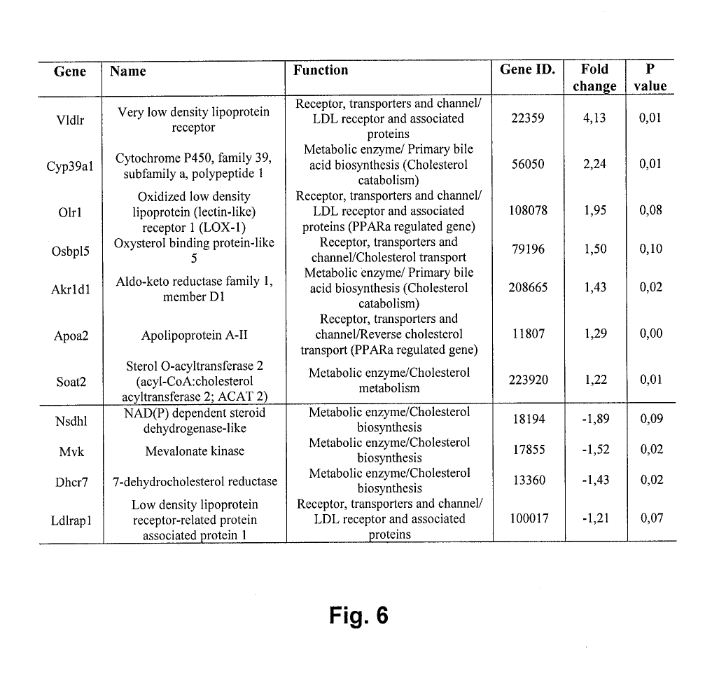

For the liver, a total of 11 genes were differentially expressed in the MPM-treated group of C57BI/6J mice as compared to their water-treated counterpart. Of these, 7 genes were up regulated while 4 genes were down regulated (FIG. 6). As compared to the WAT, fewer genes were either up or down regulated in the liver and fold changes obtained were smaller. Among up regulated genes, the great majority are involved in either lipoproteins uptake and cholesterol catabolism via bile acid synthesis. Conversely, 3 out of the 4 genes which exhibit repressed activity are linked to Cholesterol biosynthesis.

For comparison, the gavage load used during both the DIO and weight loss phase would correspond to a daily dose at the human level (70 kg) between 4 to 9 g of MPM (2 to 4 g of whey proteins).

During LF-feeding induced weight loss, MPM-treated animals tended to loose more weight, especially fat mass. Results gained from the differential gene expression pattern of adipocytes and liver from MPM-treated mice at the end of the weight loss phase bring also many insights about the mechanism of action of MPM in body weight management.

After 24 days following switching from HF to the LF diet, both MPM- and Water-treated mice were in stable condition while both body weight and food intake were at comparable levels from the last 7 days prior to sacrifice and organs removal. The difference in gene expression obtained in this experiment is thus likely attributable to the difference in the gavages treatment.

The increased expression in the epididymal fat pad of MPM-treated animals of both PPARγ is thus of particular interest since the adipose tissue ability to expand and properly store lipids may contribute to reduce insulin resistance and the metabolic complications that develop during obesity. Thiazolidinediones (TZDs) are well-known PPARγ agonists and weight gain through fat mass accretion appear to be one distinctive side effect of these agents in both humans and rodents. More importantly however is that PPARγ activation would result in WAT redistribution in favour of more peripheral fat and less visceral fat. PPARγ activation and expression is important in the presence of excess lipids, enabling the hyperplasic (increase in numbers) instead of the hypertrophic (increase in size) response of adipose tissue that retain their ability to store fat and reduce the overflow of free fatty acids to peripheral tissues (lipotoxicity) thus promoting maintenance of an adequate insulin sensitivity. Concomitantly, the plasma TG tend to increase during 8 weeks of HF feeding of MPM treated mice as compared to the water-treated control like the PPARγ agonistic effect of with some TZDs.

In the liver, it is known in the art that a caloric restriction tended to inhibit pathways involved in lipids biosynthesis, which is accordance to the present results.

DIO-057BI/6J treated with a daily oral gavage of MPM and submitted to a caloric restriction procedure during 24 days, loose more weight as compared to their water- and skim-milk-treated counterparts. It was concomitantly shown that MPM-treated WAT exhibited up-regulation of PPARγ. MPM accentuate and shorten the gene activation cascade observed during caloric restriction-induced weight loss.

### Example 7

### Double-Blind, Placebo-Controlled, Randomized, Parallel-Group, Multi-Center Clinical Trial with MPM to Assess the Rate of Normalization of Lipid Profiles in Individuals with High Cholesterol Levels

The clinical trial was carried out by a Contract Research Organization and followed the highest clinical standards (GCPs) as a randomized, double-blind, conform parallel groups (placebo-controlled) and multi-centered. The objective of the trial was to evaluate the safety and efficacy of MPM over a 12-week duration with 3 visits. The trial was conducted in Germany on a total of 161 randomized participants (47% in hospital lipid outpatient clinic and 53% general practitioners) for whom the diet was not controlled (free-living). The volunteer study participants were given bid either 15 g MPM or an isoproteic, carbohydrate, calcium placebo provided as a ready-to-mix powder juice formula to be prepared at home.

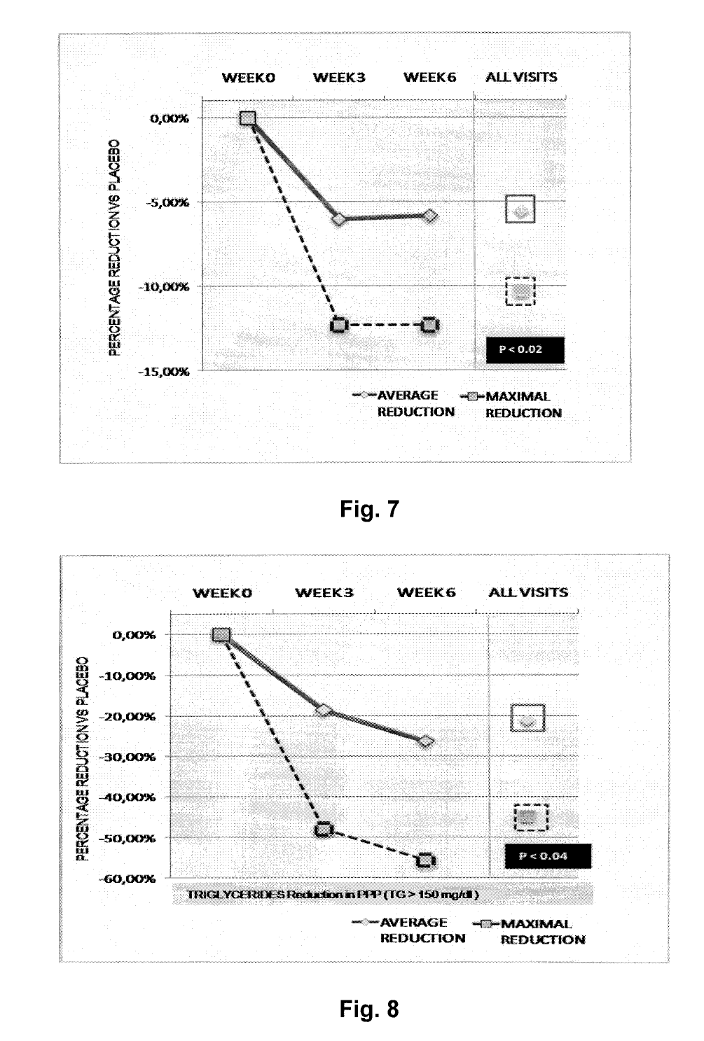

The trial demonstrated that MPM was beneficial for all study participants (131 participants completed the entire trial) and showed an significant impact on circulating glucose and blood lipid levels. The positive impact seen on Total-C, LDL-C and lipid ratios depended on the type of medical setting into which participants were monitored. Study participants randomized in a hospital lipid outpatient clinic had their LDL-C decreased compared to baseline by an average of 6% and up to 12% for maximum responders (p=0.04 vs placebo) after 12 weeks (FIG. 7). This reduction was rapidly achieved only after 3 weeks and maintained throughout the study (p<0.001 vs placebo).

There were also significant impacts on TGs (p=0.004) and HbA1c (p=0.03) for all study participants. Study participants with both, high LDL-C and TGs (>150 mg/dL), saw their TGs strikingly reduced by an average of 20% and reaching 50% for maximum responses (FIG. 8). Key lipid secondary endpoints such as Total-C (p=0.002 vs placebo), ratios of Total-C/HDL-C (p=0.01 vs placebo) and LDL-C/HDL-C (p=0.02 vs placebo) were also positively impacted. Finally, relative LDL-C reduction over 12 weeks was greater for volunteer participants with a body morphogenic index (BMI) above 25 Kg/m² thus suggesting that MPM is likely to be useful for individuals with an overweight condition.

It is important to note that study participants having impaired fasting glucose (between 100-125 mg/dL) benefited from MPM since significant effect was seen for HbA1c (p=0.05). This significant result was achieved although this subgroup did not exhibit high HbA1c value at baseline (5.9%).

Altogether, these results suggest an important metabolic impact of MPM on a variety of markers associated with increased CV risks and obesity and deserves further investigation for metabolic syndrome and diabetes. Moreover, analysis of body morphogenic animal experiments indicated a trend towards improvement of lean body mass suggesting that MPM favors a better balance between fat and muscle thus making it useful product in a context of weight management.

### Example 8

### Double-Blind, Placebo-Controlled, Randomized, Parallel-Groups, Multi-Center Clinical Trial for the Evaluation of Beneficial Effects of MPM in Comparison to Placebo as a Supportive Measure in the Management of Mild Forms of Metabolic Syndrome

MPM was tested in a large, multi-centered, randomized, double-blind, placebo-controlled, parallel group study. The study was conducted by a CRO in Germany according to GCP guidelines. The objective of the trial was to evaluate the safety and efficacy of MPM over a 12-week duration with 3 visits.

The trial designed to assess the hypothesis that MPM exerts multiple metabolic impact in humans exhibiting the metabolic syndrome was positive. All participants assigned to the MPM group benefited from MPM in either maintaining or improving metabolic health in contrast to the placebo.

Two hundred participants diagnosed and exhibiting high triglyceride (TG) levels (>150 mg/dL) combined to at least 2 other features defining the syndrome (low HDL, abdominal obesity, high blood pressure and high fasting blood glucose) were included in the trial and randomized to take 7 gr. bid of MPM in a spoonable yogurt format for three months. In the end, the patient disposition was excellent and in this respect, the trial was well run according to protocol, the randomization was effective with well balanced groups at baseline.

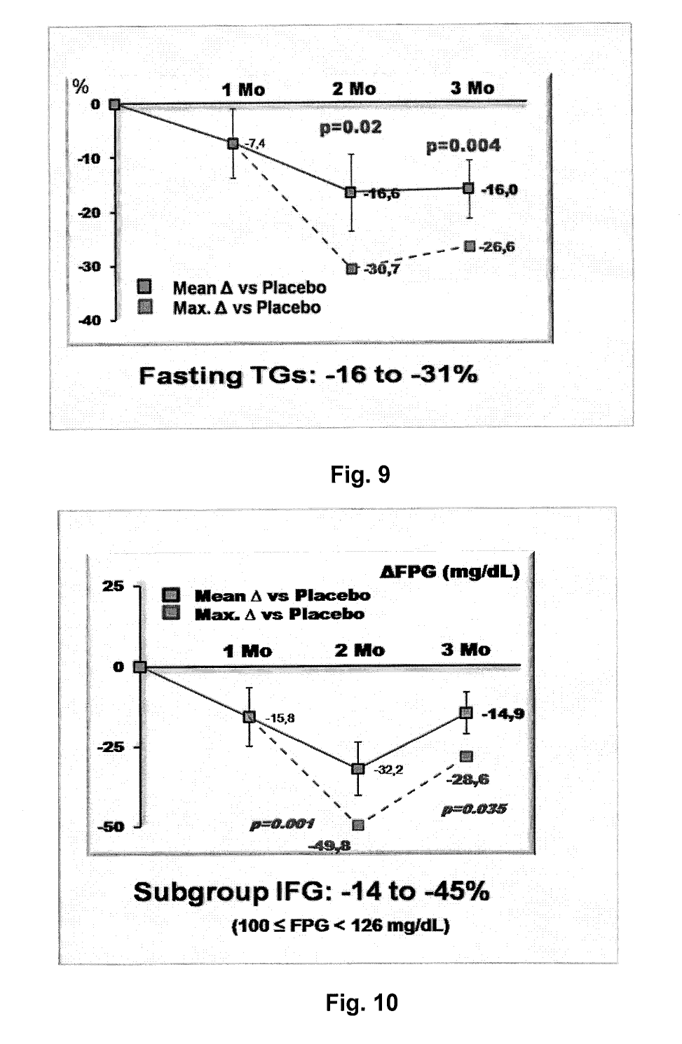

The primary end-point (TG) was met which means that a significant TG reduction (p<0.007) by treatment was detected when all patients are considered (ITT population) (an average reduction of −15% vs placebo) (FIG. 9). In addition, the trial data review revealed that the MPM provide with a positive and beneficial impact on main features defining the metabolic syndrome such as an improved fasting glucose level (FIG. 10), body weight and systolic blood pressure. It is important to note that during the course of this study, no notable safety problems were recorded in the active MPM group and the investigational product was rated as well-tolerated by most of the study participants.

While the disclosure has been described in connection with specific embodiments thereof, it will be understood that it is capable of further modifications and this application is intended to cover any variations, uses, or adaptations of the disclosure following, in general, the principles of the disclosure and including such departures from the present disclosure as come within known or customary practice within the art to which the invention pertains and as may be applied to the essential features hereinbefore set forth, and as follows in the scope of the appended claims.

1. A method of modulating peroxisome proliferator activated receptor (PPAR) activity in a patient comprising administering to said subject an effective amount of a malleable protein matrix (MPM).

2. The method of claim 1, wherein said MPM is a whey proteins hydrolysate as defined by Chemical Abstract Service No. 308074-13-7.

3. The method of claim 1, wherein PPAR activity is at least one of PPARα activity, PPARδ activity or PPARγ activity.

4. The method of claim 1, wherein said modulation of PPAR activity increase the amount of free fatty acids in the patient.

5. The method of claim 1, wherein said modulation of PPAR activity stabilizes blood lipids and glucose levels in the patient.

6. The method of claim 1, wherein said modulation of PPAR activity reduces plasma triglycerides levels in the patient.

7. The method of claim 1, wherein said modulation of PPAR activity reduces cholesterols levels in the patient.

8. The method of claim 1, wherein said MPM is administered concurrently with another therapeutic agent.

9. The method of claim 8, wherein said therapeutic agent is a cytokine, a steroid, a chemotherapeutic agent, an antibiotic, a radiation, an antiobesity agent, an appetite regulating agent, an antidiabetic agent or an antihypertensive agent.

10-17. (canceled)

18. A pharmaceutical composition for modulating a peroxisome proliferator activated receptor (PPAR) activity comprising a therapeutically effective amount of a malleable protein matrix (MPM) and a pharmaceutically acceptable excipient.

19. The composition of claim 18, wherein said MPM is a whey proteins hydrolysate as defined by Chemical Abstract Service No. 308074-13-7.

20. The composition of claim 18, wherein PPAR activity is at least one of PPARα activity, PPARδ activity or PPARγ activity.

21. The composition of claim 18, further comprising a therapeutic agent.

22. The composition of claim 21, wherein said therapeutic agent is a cytokine, a steroid, a chemotherapeutic agent, an antibiotic, a radiation, an antiobesity agent, an appetite regulating agent, an antidiabetic agent or an antihypertensive agent.

23. The composition of claim 19, wherein said composition is formulated as a medicament, a dietary supplement, a nutraceutical or a functional food.
